(19) United States
(12) Patent Application Publication
(10) Pub. No.: US 2010/0197692 A1
(43) Pub. Date: Aug. 5, 2010

(54) BICYCLIC HETEROAROMATIC COMPOUNDS AS INHIBITORS OF STEAROYL-COENZYME A DELTA-9 DESATURASE

(75) Inventors: Yeeman K. Ramtohul, Pierrefonds (CA); Chun Sing Li, Dollard-des-Ormeaux (CA); Jean-Philippe Leclerc, Laval (CA)

Correspondence Address: MERCK, P O BOX 2000, RAHWAY, NJ, 07065-0907, US

(73) Assignee: Merck Frosst Canada Ltd.

(21) Appl. No.: 12/668,537

(22) PCT Filed: Jul. 18, 2008
(86) PCT No.: PCT/CA2008/001331

§ 371 Date: Jan. 11, 2010

Related U.S. Application Data
(60) Provisional application No. 60/961,357, filed on Jul. 20, 2007.

(51) Int. Cl. A61K 31/4985; C07D 487/04; A61K 31/519; C07D 471/04; A61P 3/10; A61P 3/04; A61P 9/10

(52) U.S. Cl. 514/249; 544/254; 514/261.1; 544/255; 514/260.1; 544/350

(57) ABSTRACT

Bicyclic heteroaromatic compounds of structural formula I are inhibitors of stearoyl-coenzyme A delta-9 desaturase (SCD). The compounds of the present invention are useful for the prevention and treatment of conditions related to abnormal lipid synthesis and metabolism, including cardiovascular disease; atherosclerosis; obesity; diabetes; neurological disease; metabolic syndrome; insulin resistance; liver steatosis; and non-alcoholic steatohepatitis.

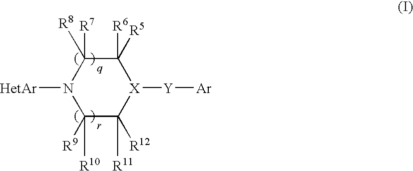

### FIELD OF THE INVENTION

The present invention relates to bicyclic heteroaromatic compounds which are inhibitors of stearoyl-coenzyme A delta-9 desaturase (SCD) and the use of such compounds to control, prevent and/or treat conditions or diseases mediated by SCD activity. The compounds of the present invention are useful for the control, prevention and treatment of conditions and diseases related to abnormal lipid synthesis and metabolism, including cardiovascular disease; atherosclerosis; obesity; diabetes; neurological disease; metabolic syndrome; insulin resistance; cancer; liver steatosis; and non-alcoholic steatohepatitis.

### BACKGROUND OF THE INVENTION

At least three classes of fatty acyl-coenzyme A (CoA) desaturases (delta-5, delta-6 and delta-9 desaturases) are responsible for the formation of double bonds in mono- and polyunsaturated fatty acyl-CoAs derived from either dietary sources or de novo synthesis in mammals. The delta-9 specific stearoyl-CoA desaturases (SCD's) catalyze the rate-limiting formation of the cis-double bond at the C9-C10 position in monounsaturated fatty acyl-CoAs. The preferred substrates are stearoyl-CoA and palmitoyl-CoA, with the resulting oleoyl and palmitoleoyl-CoA as the main components in the biosynthesis of phospholipids, triglycerides, cholesterol esters and wax esters (Dobrzyn and Natami, Obesity Reviews, 6: 169-174 (2005)).

The rat liver microsomal SCD protein was first isolated and characterized in 1974 (Strittmatter et al., PNAS, 71: 4565-4569 (1974)). A number of mammalian SCD genes have since been cloned and studied from various species. For example, two genes have been identified from rat (SCD1 and SCD2, Thiede et al., J. Biol. Chem., 261, 13230-13235 (1986)), Mihara, K., J. Biochem. (Tokyo), 108: 1022-1029 (1990)); four genes from mouse (SCD1, SCD2, SCD3 and SCD4) (Miyazaki et al., J. Biol. Chem., 278: 33904-33911 (2003)); and two genes from human (SCD1 and ACOD4 (SCD2 or SCD5)), (Zhang, et al., Biochem. J., 340: 255-264 (1991); Beiraghi, et al., Gene, 309: 11-21 (2003); Zhang et al., Biochem. J., 388: 135-142 (2005)). The involvement of SCD's in fatty acid metabolism has been known in rats and mice since the 1970's (Oshino, N., Arch. Biochem. Biophys., 149: 378-387 (1972)). This has been further supported by the biological studies of a) Asebia mice that carry the natural mutation in the SCD gene (Zheng et al., Nature Genetics, 23: 268-270 (1999)), b) SCD-null mice from targeted gene deletion (Ntambi, et al., PNAS, 99: 11482-11486 (2002), and c) the suppression of SCD expression during leptin-induced weight loss (Cohen et al., Science, 297: 240-243 (2002)). The potential benefits of pharmacological inhibition of SCD activity has been demonstrated with anti-sense oligonucleotide inhibitors (ASO) in mice (Jiang, et al., J. Clin. Invest., 115: 1030-1038 (2005)). ASO inhibition of SCD activity reduced fatty acid synthesis and increased fatty acid oxidation in primary mouse hepatocytes. Treatment of mice with SCD-ASOs resulted in the prevention of diet-induced obesity, reduced body adiposity, hepatomegaly, steatosis, postprandial plasma insulin and glucose levels, reduced de novo fatty acid synthesis, decreased the expression of lipogenic genes, and increased the expression of genes promoting energy expenditure in liver and adipose tissues. SCD knock-out mice (−/−) are characterized by reduced adiposity and increased energy expenditure. Thus, SCD inhibition represents a novel therapeutic strategy in the treatment of Type 2 diabetes, obesity, and related metabolic disorders, such as the Metabolic Syndrome.

There is compelling evidence to support that elevated SCD activity in humans is directly implicated in several common disease processes. For example, there is an elevated hepatic lipogenesis to triglyceride secretion in non-alcoholic fatty liver disease patients (Diraison, et al., Diabetes Metabolism, 29: 478-485 (2003)); Donnelly, et al., J. Clin. Invest., 115: 1343-1351 (2005)). The postprandial de novo lipogenesis is significantly elevated in obese subjects (Marques-Lopes, et al., American Journal of Clinical Nutrition, 73: 252-261 (2001)). There is a significant correlation between a high SCD activity and an increased cardiovascular risk profile including elevated plasma triglycerides, a high body mass index and reduced plasma HDL (Attie, et al., J. Lipid Res., 43: 1899-1907 (2002)). SCD activity plays a key role in controlling the proliferation and survival of human transformed cells (Scaglia and Igal, J. Biol. Chem., (2005)).

Other than the above mentioned anti-sense oligonucleotides, inhibitors of SCD activity include non-selective thia-fatty acid substrate analogs [B. Behrouzian and P. H. Buist, Prostaglandins, Leukotrienes, and Essential Fatty Acids, 68: 107-112 (2003)], cyclopropenoid fatty acids (Raju and Reiser, J. Biol. Chem., 242: 379-384 (1967)), certain conjugated long-chain fatty acid isomers (Park, et al., Biochim. Biophys. Acta, 1486: 285-292 (2000)), and a series of heterocyclic derivatives disclosed in published international patent application publications: WO 2005/011653; WO 2005/011654; WO 2005/011656; WO 2005/011657; WO 2006/014168; WO 2006/034279; WO 2006/034312; WO 2006/034315; WO 2006/034338; WO 2006/034341; WO 2006/034440; WO 2006/034441; WO 2006/034446; WO 2006/086445; WO 2006/086447; WO 2006/101521; WO 2006/125178; WO 2006/125179; WO 2006/125180; WO 2006/125181; WO 2006/125194; WO 2007/044085; WO 2007/046867; WO 2007/046868; WO 2007/050124; WO 2007/130075; and WO 2007/136746, all assigned to Xenon Pharmaceuticals, Inc. A number of international patent applications assigned to Merck Frosst Canada Ltd. that disclose SCD inhibitors useful for the treatment of obesity and Type 2 diabetes have also published: WO 2006/130986 (14 Dec. 2006); WO 2007/009236 (25 Jan. 2007); WO 2007/038865 (12 Apr. 2007); WO 2007/056846 (24 May 2007); WO 2007/071023 (28 Jun. 2007); WO 2007/134457 (29 Nov. 2007); WO 2007/143823 (21 Dec. 2007); and WO 2007/143824 (21 Dec. 2007). WO 2008/003753 (assigned to Novartis) discloses a series of pyrazolo[1,5-a]pyrimidine analogs as SCD inhibitors, and WO 2007/143597 (assigned to Novartis and Xenon Pharmaceuticals) discloses heterocyclic derivatives as SCD inhibitors. Small molecule SCD inhibitors have also been described by G. Liu, et al., “Discovery of Potent, Selective, Orally Bioavailable SCD1 Inhibitors,” in J. Med. Chem., 50: 3086-3100 (2007) and by H. Zhao, et al., “Discovery of 1-(4-phenoxypiperidin-1-yl)-2-arylaminoethanone SCD 1 inhibitors,” Bioorg. Med. Chem. Lett., 17: 3388-3391 (2007).

The present invention is concerned with novel heteroaromatic compounds as inhibitors of stearoyl-CoA delta-9 desaturase which are useful in the treatment and/or prevention of various conditions and diseases mediated by SCD activity including those related, but not limited, to elevated lipid levels, as exemplified in non-alcoholic fatty liver disease, cardiovascular disease, obesity, hyperglycemia, Type 2 diabetes, Metabolic Syndrome, and insulin resistance.

The role of stearoyl-coenzyme A desaturase in lipid metabolism has been described by M. Miyazaki and J. M. Ntambi, Prostaglandins, Leukotrienes, and Essential Fatty Acids, 68: 113-121 (2003). The therapeutic potential of the pharmacological manipulation of SCD activity has been described by A. Dobryzn and J. M. Ntambi, in “Stearoyl-CoA desaturase as a new drug target for obesity treatment,” Obesity Reviews, 6: 169-174 (2005).

### SUMMARY OF THE INVENTION

The present invention relates to bicyclic heteroaromatic compounds of structural formula I:

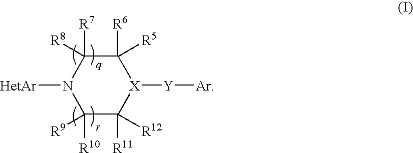

These bicyclic heteroaromatic compounds are effective as inhibitors of SCD. They are therefore useful for the treatment, control or prevention of disorders responsive to the inhibition of SCD, such as diabetes, insulin resistance, lipid disorders, obesity, atherosclerosis, and metabolic syndrome.

The present invention also relates to pharmaceutical compositions comprising the compounds of the present invention and a pharmaceutically acceptable carrier.

The present invention also relates to methods for the treatment, control, or prevention of disorders, diseases, or conditions responsive to inhibition of SCD in a subject in need thereof by administering the compounds and pharmaceutical compositions of the present invention.

The present invention also relates to methods for the treatment, control, or prevention of Type 2 diabetes, insulin resistance, obesity, lipid disorders, atherosclerosis, and metabolic syndrome by administering the compounds and pharmaceutical compositions of the present invention.

The present invention also relates to methods for the treatment, control, or prevention of obesity by administering the compounds of the present invention in combination with a therapeutically effective amount of another agent known to be useful to treat the condition.

The present invention also relates to methods for the treatment, control, or prevention of Type 2 diabetes by administering the compounds of the present invention in combination with a therapeutically effective amount of another agent known to be useful to treat the condition.

The present invention also relates to methods for the treatment, control, or prevention of atherosclerosis by administering the compounds of the present invention in combination with a therapeutically effective amount of another agent known to be useful to treat the condition.

The present invention also relates to methods for the treatment, control, or prevention of lipid disorders by administering the compounds of the present invention in combination with a therapeutically effective amount of another agent known to be useful to treat the condition.

The present invention also relates to methods for treating metabolic syndrome by administering the compounds of the present invention in combination with a therapeutically effective amount of another agent known to be useful to treat the condition.

### DETAILED DESCRIPTION OF THE INVENTION

The present invention is concerned with bicyclic heteroaromatic compounds useful as inhibitors of SCD. Compounds of the present invention are described by structural formula I:

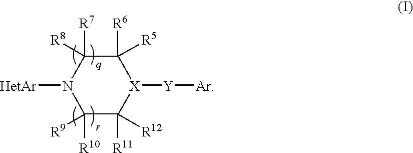

and pharmaceutically acceptable salts thereof; wherein
HetAr is a fused heteroaromatic ring selected from the group consisting of:

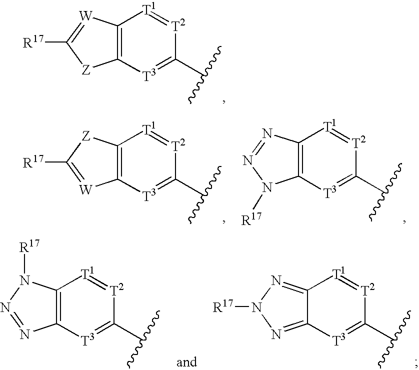

wherein W is N or CR¹⁶;
Z is O, S, or NR¹⁵;
T¹, T², and T³ are each independently N or CR¹⁶, with the proviso that at least one of T¹, T², and T³ is N;
q is 0 or 1;
r is 0 or 1;
X—Y is N—C(O), CR¹⁴—O, CR¹⁴—S(O)₀₋₂, or CR¹³—CR¹R²;
Ar is phenyl, naphthyl, or heteroaryl optionally substituted with one to five R³ substituents;
R¹ and R² are each independently hydrogen or C₁₋₃ alkyl, wherein alkyl is optionally substituted with one to three substituents independently selected from fluorine and hydroxy;
each R³ is independently selected from the group consisting of:

C₁₋₆ alkyl,

C₂₋₆ alkenyl,

(CH₂)ₙ-phenyl,

(CH₂)ₙ-naphthyl,

(CH₂)ₙ-heteroaryl,

(CH₂)ₙ-heterocyclyl,

(CH₂)ₙC₃-₇ cycloalkyl,

halogen,

nitro,

(CH₂)ₙOR⁴,

(CH₂)ₙN(R⁴)₂,

(CH₂)ₙC≡N,

(CH₂)ₙCO₂R⁴,

(CH₂)ₙNR⁴SO₂R⁴

(CH₂)ₙSO₂N(R⁴)₂,

(CH₂)ₙS(O)₀₋₂R⁴,

(CH₂)ₙNR⁴C(O)N(R⁴)₂,

(CH₂)ₙC(O)N(R⁴)₂,

(CH₂)ₙNR⁴C(O)R⁴,

(CH₂)ₙNR⁴CO₂R⁴,

(CH₂)ₙC(O)R⁴,

O(CH₂)ₙC(O)N(R⁴)₂,

(CH₂)ₛ—Z—(CH₂)ₜ-phenyl,

(CH₂)ₛ—Z—(CH₂)ₜ-naphthyl,

(CH₂)ₛ—Z—(CH₂)ₜ-heteroaryl,

(CH₂)ₛ—Z—(CH₂)ₜ-heterocyclyl,

(CH₂)ₛ—Z—(CH₂)ₜ—C₃₋₇ cycloalkyl,

(CH₂)ₛ—Z—(CH₂)ₜ—OR⁴,

(CH₂)ₛ—Z—(CH₂)ₜ—N(R⁴)₂,

(CH₂)ₛ—Z—(CH₂)ₜ—NR⁴SO₂R⁴,

(CH₂)ₛ—Z—(CH₂)ₜ—C≡N,

(CH₂)ₛ—Z—(CH₂)ₜ—CO₂R⁴,

(CH₂)ₛ—Z—(CH₂)ₜ—SO₂N(R⁴)₂,

(CH₂)ₛ—Z—(CH₂)ₜ—S(O)₀₋₂R⁴,

(CH₂)ₛ—Z—(CH₂)ₜ—NR⁴C(O)N(R⁴)₂,

(CH₂)ₛ—Z—(CH₂)ₜ—C(O)N(R⁴)₂,

(CH₂)ₛ—Z—(CH₂)ₜ—NR⁴C(O)R⁴,

(CH₂)ₛ—Z—(CH₂)ₜ—NR⁴CO₂R⁴,

(CH₂)ₛ—Z—(CH₂)ₜ—C(O)R⁴,

CF₃,

CH₂CF₃,

OCF₃, and

OCH₂CF₃;
in which phenyl, naphthyl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy; and wherein any methylene (CH₂) carbon atom in R³ is optionally substituted with one to two groups independently selected from fluorine, hydroxy, and C₁₋₄ alkyl; or two substituents when on the same methylene (CH₂) group are taken together with the carbon atom to which they are attached to form a cyclopropyl group;
Z is O, S, or NR⁴;
each R⁴ is independently selected from the group consisting of

hydrogen,

C₁₋₆ alkyl,

(CH₂)ₘ-phenyl,

(CH₂)ₘ-heteroaryl,

(CH₂)ₘ-naphthyl, and

(CH₂)ₘC₃-₇ cycloalkyl;
wherein alkyl, phenyl, heteroaryl, and cycloalkyl are optionally substituted with one to three groups independently selected from halogen, C₁₋₄ alkyl, and C₁₋₄ alkoxy; or two R⁴ groups together with the atom to which they are attached form a 4- to 8-membered mono- or bicyclic ring system optionally containing an additional heteroatom selected from O, S, NH, and NC₁₋₄ alkyl;
R⁵, R⁶, R⁷, R⁸, R⁹, R¹⁰, R¹¹, and R¹² are each independently hydrogen, fluorine, or C₁₋₃ alkyl, wherein alkyl is optionally substituted with one to three substituents independently selected from fluorine and hydroxy;
R¹³ is hydrogen, C₁₋₃ alkyl, fluorine, or hydroxy;
each R¹⁴ is hydrogen or C₁₋₃ alkyl;
R¹⁵ is selected from the group consisting of hydrogen, C₁₋₄ alkyl, C₁₋₄ alkylcarbonyl, aryl-C₁₋₂ alkylcarbonyl, arylcarbonyl, C₁₋₄ alkylaminocarbonyl, C₁₋₄ alkylsulfonyl, arylsulfonyl, aryl-C₁₋₂ alkylsulfonyl, C₁₋₄ alkyloxycarbonyl, aryloxycarbonyl, and aryl-C₁₋₂ alkyloxycarbonyl;
R¹⁶ is hydrogen, amino, halogen, or C₁₋₃ alkyl optionally substituted with one to five fluorines;
R¹⁷ is selected from the group consisting of:

—(CH₂)ᵥC(O)R^{a};

—O(CH₂)_{w}C(O)R^{a},

—S(CH₂)_{w}C(O)R^{a},

—NH(CH₂)_{w}C(O)R^{a},

—NCH₃(CH₂)_{w}C(O)R^{a},

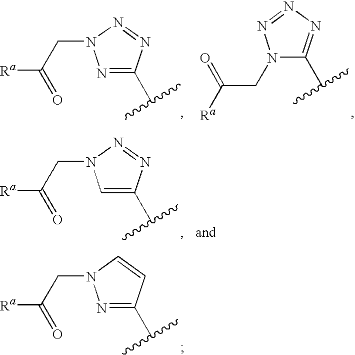

R^{a} is —OH, —OC₁₋₄ alkyl, —NH₂, —NHSO₂C₁₋₄ alkyl, —NHSO₂C₃₋₆ cycloalkyl, or —NHSO₂CH₂C₃₋₆ cycloalkyl;
each m is independently an integer from 0 to 2;
each n is independently an integer from 0 to 2;
each s is independently an integer from 1 to 3;
each t is independently an integer from 1 to 3;
v is an integer from 1 to 3; and
each w is an integer from 1 to 2.

In one embodiment of the compounds of the present invention, q and r are each 1, affording a 6-membered piperidine ring.

In a second embodiment of the compounds of the present invention, q is 1 and r is 0, affording a 5-membered pyrrolidine ring.

In a third embodiment of the compounds of the present invention, q and r are each 0, affording a 4-membered azetidine ring.

In a fourth embodiment of the compounds of the present invention, X—Y is N—C(O). In a class of this embodiment, Ar is phenyl substituted with one to three R³ substituents as defined above.

In a fifth embodiment of the compounds of the present invention, X—Y is CR¹⁴—O. In a class of this embodiment, R¹⁴ is hydrogen and Ar is phenyl substituted with one to three R³ substituents as defined above.

In a sixth embodiment of the compounds of the present invention, X—Y is CR¹⁴—S. In a class of this embodiment, R¹⁴ is hydrogen and Ar is phenyl substituted with one to three R³ substituents as defined above.

In a seventh embodiment of the compounds of the present invention, X—Y is CR¹³—CR¹R². In a class of this embodiment, R¹, R², and R¹³ are each hydrogen and Ar is phenyl substituted with one to three R³ substituents as defined above.

In an eighth embodiment of the compounds of the present invention, R⁵, R⁶, R⁷, R⁸, R⁹, R¹⁰, R¹¹, and R¹² are each hydrogen.

In a ninth embodiment of the compounds of the present invention, T¹ is CR¹⁶, and T² and T³ are each N; or T² is CR¹⁶, and T¹ and T³ are each N. In a class of this embodiment, R¹⁶ is hydrogen.

In a tenth embodiment of the compounds of the present invention, HetAr is

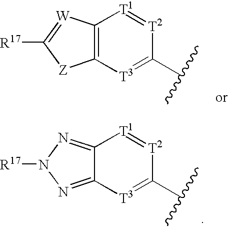

wherein T¹, T², T³, and R¹⁷ are as defined above. In a class of this embodiment, T¹ is CH; and T² and T³ are each N. In another class of this embodiment, T² is CH; and T¹ and T³ are each N In a subclass of each class of this embodiment, Z is S, and W is NH.

In an eleventh embodiment of the compounds of the present invention, R^{a} is OH or —OC₁₋₄ alkyl. In a class of this embodiment, v is 2 and each w is 1.

In a twelfth embodiment of the compounds of the present invention, Ar is phenyl substituted with one to two substituents independently selected from the group consisting from C₁₋₄ alkyl, halogen, and CF₃.

In a thirteenth embodiment of the compounds of the present invention,
X—Y is CH—O;
q and r are each 1;
R⁵, R⁶, R⁷, R⁸, R⁹, R¹⁰, R¹¹, and R¹² are each hydrogen;
Ar is phenyl substituted with one to three R³ substituents as defined above;
HetAr is

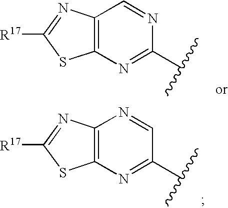

R¹⁷ is selected from the group consisting of:

—(CH₂)₂C(O)R^{a};

—OCH₂C(O)R^{a},

—SCH₂C(O)R^{a},

—NHCH₂C(O)R^{a}, and

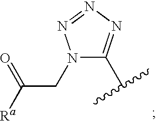

and
R^{a} is —OH or —OC₁₋₄ alkyl.

In a class of this embodiment, Ar is phenyl substituted with one to two substituents independently selected from the group consisting from C₁₋₄ alkyl, halogen, and CF₃.

Illustrative, but nonlimiting examples, of compounds of the present invention that are useful as inhibitors of SCD are the following:

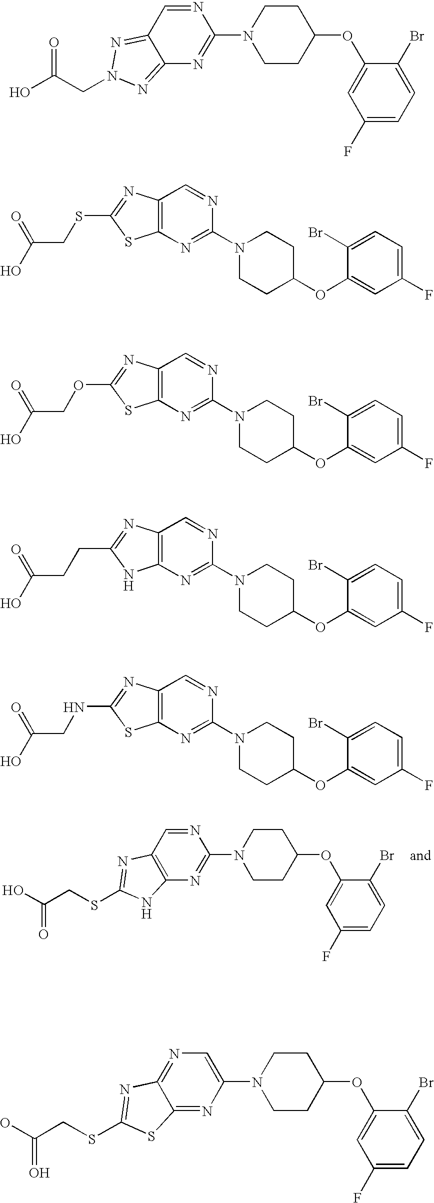

and pharmaceutically acceptable salts thereof.

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃-₁₀, the term alkyl also includes cycloalkyl groups, and combinations of linear or branched alkyl chains combined with cycloalkyl structures. When no number of carbon atoms is specified, C₁₋₆ is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5(6H)-one-3-yl, and the like.

“Heteroaryl” means an aromatic or partially aromatic heterocycle that contains at least one ring heteroatom selected from O, S and N. “Heteroaryl” thus includes heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

Compounds of structural formula I may contain one or more asymmetric centers and can thus occur as racemates and racemic mixtures, single enantiomers, diastereomeric mixtures and individual diastereomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present invention. An example of tautomers which are intended to be encompassed within the compounds of the present invention are illustrated below:

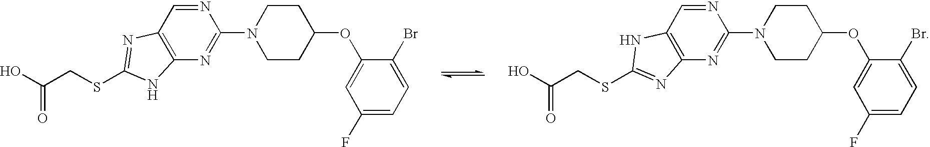

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural formula I are included in the present invention as well.

The subject compounds are useful in a method of inhibiting the stearoyl-coenzyme A delta-9 desaturase enzyme (SCD) in a patient such as a mammal in need of such inhibition comprising the administration of an effective amount of the compound. The compounds of the present invention are therefore useful to control, prevent, and/or treat conditions and diseases mediated by high or abnormal SCD enzyme activity.

Thus, one aspect of the present invention concerns a method of treating hyperglycemia, diabetes or insulin resistance in a mammalian patient in need of such treatment, which comprises administering to said patient an effective amount of a compound in accordance with structural formula I or a pharmaceutically salt or solvate thereof.

A second aspect of the present invention concerns a method of treating non-insulin dependent diabetes mellitus (Type 2 diabetes) in a mammalian patient in need of such treatment comprising administering to the patient an antidiabetic effective amount of a compound in accordance with structural formula I.

A third aspect of the present invention concerns a method of treating obesity in a mammalian patient in need of such treatment comprising administering to said patient a compound in accordance with structural formula I in an amount that is effective to treat obesity.

A fourth aspect of the invention concerns a method of treating metabolic syndrome and its sequelae in a mammalian patient in need of such treatment comprising administering to said patient a compound in accordance with structural formula I in an amount that is effective to treat metabolic syndrome and its sequelae. The sequelae of the metabolic syndrome include hypertension, elevated blood glucose levels, high triglycerides, and low levels of HDL cholesterol.

A fifth aspect of the invention concerns a method of treating a lipid disorder selected from the group consisting of dyslipidemia, hyperlipidemia, hypertriglyceridemia, hypercholesterolemia, low HDL and high LDL in a mammalian patient in need of such treatment comprising administering to said patient a compound in accordance with structural formula I in an amount that is effective to treat said lipid disorder.

A sixth aspect of the invention concerns a method of treating atherosclerosis in a mammalian patient in need of such treatment comprising administering to said patient a compound in accordance with structural formula I in an amount effective to treat atherosclerosis.

A seventh aspect of the invention concerns a method of treating cancer in a mammalian patient in need of such treatment comprising administering to said patient a compound in accordance with structural formula I in an amount effective to treat cancer. In one embodiment of this aspect of the invention, the cancer is liver cancer.

A further aspect of the invention concerns a method of treating a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) metabolic syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to treat said condition.

Yet a further aspect of the invention concerns a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) metabolic syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to delay the onset of said condition.

Yet a further aspect of the invention concerns a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) metabolic syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to reduce the risk of developing said condition.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, species can be treated. However, the method can also be practiced in other species, such as avian species (e.g., chickens).

The present invention is further directed to a method for the manufacture of a medicament for inhibiting stearoyl-coenzyme A delta-9 desaturase enzyme activity in humans and animals comprising combining a compound of the present invention with a pharmaceutically acceptable carrier or diluent. More particularly, the present invention is directed to the use of a compound of structural formula I in the manufacture of a medicament for use in treating a condition selected from the group consisting of hyperglycemia, Type 2 diabetes, insulin resistance, obesity, and a lipid disorder in a mammal, wherein the lipid disorder is selected from the group consisting of dyslipidemia, hyperlipidemia, hypertriglyceridemia, hypercholesterolemia, low HDL, and high LDL.

The subject treated in the present methods is generally a mammal, preferably a human being, male or female, in whom inhibition of stearoyl-coenzyme A delta-9 desaturase enzyme activity is desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibitors of stearoyl-coenzyme A delta-9 desaturase (SCD) enzyme activity may be demonstrated by the following microsomal and whole-cell based assays:

### I. SCD-Induced Rat Liver Microsome Assay:

The activity of compounds of formula I against the SCD enzyme is determined by following the conversion of radiolabeled-stearoyl-CoA to oleoyl-CoA using SCD-induced rat liver microsome and a previously published procedure with some modifications (Joshi, et al., J. Lipid Res., 18: 32-36 (1977)). After feeding wistar rats with a high carbohydrate/fat-free rodent diet (LabDiet #5803, Purina) for 3 days, the SCD-induced livers were homogenized (1:10 w/v) in 250 mM sucrose, 1 mM EDTA, 5 mM DTT and 50 mM Tris-HCl (pH 7.5). After a 20 min centrifugation (18,000×g/4° C.) to remove tissue and cell debris, the microsome was prepared by a 100,000×g centrifugation (60 min) with the resulting pellet suspended in 100 mM sodium phosphate, 20% glycerol and 2 mM DTT. Test compound in 2 μL DMSO was incubated for 15 min. at room temperature with 180 μL of the microsome (typically at about 100 μg/mL, in Tris-HCl buffer (100 mM, pH 7.5), ATP (5 mM), Coenzyme A (0.1 mM), Triton X-100 (0.5 mM) and NADH (2 mM)). The reaction was initiated by the addition of 20 μL of [³H]-Stearoyl-CoA (final concentration at 2 μM with the radioactivity concentration at 1 μCi/mL), and terminated by the addition of 150 μL of 1N sodium hydroxide. After 60 min at room temperature to hydrolyze the oleoyl-CoA and stearoyl-CoA, the solution was acidified by the addition of 150 μL of 15% phosphoric acid (v/v) in ethanol supplemented with 0.5 mg/mL stearic acid and 0.5 mg/mL oleic acid. [³H]-oleic acid and [³H]-stearic acid were then quantified on a HPLC that is equipped with a C-18 reverse phase column and a Packard Flow Scintillation Analyzer. Alternatively, the reaction mixture (80 μL) was mixed with a calcium chloride/charcoal aqueous suspension (100 μL of 15% (w/v) charcoal plus 20 μL of 2 N CaCl₂). The resulting mixture was centrifuged to precipitate the radioactive fatty acid species into a stable pellet. Tritiated water from SCD-catalyzed desaturation of 9,10-[³H]-stearoyl-CoA was quantified by counting 50 μL of the supernant on a scintillation counter.

### II. Whole Cell-Based SCD (Delta-9), Delta-5 and Delta-6 Desaturase Assays:

Human HepG2 cells were grown on 24-well plates in MEM media (Gibco cat #11095-072) supplemented with 10% heat-inactivated fetal bovine serum at 37° C. under 5% CO₂ in a humidified incubator. Test compound dissolved in the media was incubated with the subconfluent cells for 15 min at 37° C. [1-¹⁴C]-stearic acid was added to each well to a final concentration of 0.05 μCi/mL to detect SCD-catalyzed [¹⁴C]-oleic acid formation. 0.05 μCi/mL of [1-¹⁴C]-eicosatrienoic acid or [1-¹⁴C]-linolenic acid plus 10 μM of 2-amino-N-(3-chlorophenyl)benzamide (a delta-5 desaturase inhibitor) was used to index the delta-5 and delta-6 desaturase activities, respectively. After 4 h incubation at 37° C., the culture media was removed and the labeled cells were washed with PBS (3×1 mL) at room temperature. The labeled cellular lipids were hydrolyzed under nitrogen at 65° C. for 1 h using 400 μL of 2N sodium hydroxide plus 50 μL of L-α-phosphatidylcholine (2 mg/mL in isopropanol, Sigma #P-3556). After acidification with phosphoric acid (60 μL), the radioactive species were extracted with 300 μL of acetonitrile and quantified on a HPLC that was equipped with a C-18 reverse phase column and a Packard Flow Scintillation Analyzer. The levels of [¹⁴C]-oleic acid over [¹⁴C]-stearic acid, [¹⁴C]-arachidonic acid over [¹⁴C]-eicosatrienoic acid, and [¹⁴C]-eicosatetraenoic acid (8,11,14,17) over [¹⁴C]-linolenic acid were used as the corresponding activity indices of SCD, delta-5 and delta-6 desaturase, respectively.

The SCD inhibitors of formula I, particularly the inhibitors of Examples 1 through 16 exhibit an inhibition constant IC₅₀ of less than 1 μM and more typically less than 0.1 μM. Generally, the IC₅₀ ratio for delta-5 or delta-6 desaturases to SCD for a compound of formula I, particularly for Examples 1 through 16, is at least about ten or more, and preferably about hundred or more.

### In Vivo Efficacy of Compounds of the Present Invention:

The in vivo efficacy of compounds of formula I was determined by following the conversion of [1-¹⁴C]-stearic acid to [1-¹⁴C]oleic acid in animals as exemplified below. Mice were dosed with a compound of formula I and one hour later the radioactive tracer, [1-¹⁴C]-stearic acid, was dosed at 20 μCi/kg IV. At 3 h post dosing of the compound, the liver was harvested and then hydrolyzed in 10 N sodium hydroxide for 24 h at 80° C., to obtain the total liver fatty acid pool. After phosphoric acid acidification of the extract, the amount of [1-¹⁴C]-stearic acid and [1-¹⁴C]-oleic acid was quantified on a HPLC that was equipped with a C-18 reverse phase column and a Packard Flow Scintillation Analyzer.

The subject compounds are further useful in a method for the prevention or treatment of the aforementioned diseases, disorders and conditions in combination with other agents.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also include therapies in which the compound of formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of formula I, and either administered separately or in the same pharmaceutical composition, include, but are not limited to:

(a) dipeptidyl peptidase IV (DPP-IV) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (NN-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), melanin-concentrating hormone (MCH) receptor antagonists, and microsomal triglyceride transfer protein (MTP) inhibitors;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors;

(u) AMPK activators; and

(v) oxyntomodulin and derivatives and analogs thereof.

Dipeptidyl peptidase-IV inhibitors that can be combined with compounds of structural formula I include those disclosed in U.S. Pat. No. 6,699,871; WO 02/076450 (3 Oct. 2002); WO 03/004498 (16 Jan. 2003); WO 03/004496 (16 Jan. 2003); EP 1 258 476 (20 Nov. 2002); WO 02/083128 (24 Oct. 2002); WO 02/062764 (15 Aug. 2002); WO 03/000250 (3 Jan. 2003); WO 03/002530 (9 Jan. 2003); WO 03/002531 (9 Jan. 2003); WO 03/002553 (9 Jan. 2003); WO 03/002593 (9 Jan. 2003); WO 03/000180 (3 Jan. 2003); WO 03/082817 (9 Oct. 2003); WO 03/000181 (3 Jan. 2003); WO 04/007468 (22 Jan. 2004); WO 04/032836 (24 Apr. 2004); WO 04/037169 (6 May 2004); and WO 04/043940 (27 May 2004). Specific DPP-IV inhibitor compounds include sitagliptin (MK-0431); NVP-DPP-728; vildagliptin (LAF 237); P93/01; alogliptin (SYR-322); denagliptin; and saxagliptin (BMS 477118).

Antiobesity compounds that can be combined with compounds of structural formula I include fenfluramine, dexfenfluramine, phentermine, sibutramine, orlistat, neuropeptide Y₁ or Y₅ antagonists, cannabinoid CB1 receptor antagonists or inverse agonists, melanocortin receptor agonists, in particular, melanocortin-4 receptor agonists, ghrelin antagonists, bombesin receptor agonists, and melanin-concentrating hormone (MCH) receptor antagonists. For a review of anti-obesity compounds that can be combined with compounds of structural formula I, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); and J. A. Fernandez-Lopez, et al., “Pharmacological Approaches for the Treatment of Obesity,” Drugs, 62: 915-944 (2002).

Neuropeptide Y5 antagonists that can be combined with compounds of structural formula I include those disclosed in U.S. Pat. No. 6,335,345 (1 Jan. 2002) and WO 01/14376 (1 Mar. 2001); and specific compounds identified as GW 59884A; GW 569180A; LY366377; and CGP-71683A.

Cannabinoid CB1 receptor antagonists that can be combined with compounds of formula I include those disclosed in PCT Publication WO 03/007887; U.S. Pat. No. 5,624,941, such as rimonabant; PCT Publication WO 02/076949, such as SLV-319; U.S. Pat. No. 6,028,084; PCT Publication WO 98/41519; PCT Publication WO 00/10968; PCT Publication WO 99/02499; U.S. Pat. No. 5,532,237; U.S. Pat. No. 5,292,736; PCT Publication WO 03/086288; PCT Publication WO 03/087037; PCT Publication WO 04/048317; PCT Publication WO 03/007887; PCT Publication WO 03/063781; PCT Publication WO 03/075660; PCT Publication WO 03/077847; PCT Publication WO 03/082190; PCT Publication WO 03/082191; PCT Publication WO 03/087037; PCT Publication WO 03/086288; PCT Publication WO 04/012671; PCT Publication WO 04/029204; PCT Publication WO 04/040040; PCT Publication WO 01/64632; PCT Publication WO 01/64633; and PCT Publication WO 01/64634. Specific cannabinoid CB1 receptor antagonists include rimonabant and taranabant.

Melanocortin-4 receptor (MC4R) agonists useful in the present invention include, but are not limited to, those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/089307, WO 2004/078716, WO 2004/078717, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02//092566, WO 03/-093234, WO 03/095474, and WO 03/104761.

One particular aspect of combination therapy concerns a method of treating a condition selected from the group consisting of hypercholesterolemia, atherosclerosis, low HDL levels, high LDL levels, hyperlipidemia, hypertriglyceridemia, and dyslipidemia, in a mammalian patient in need of such treatment comprising administering to the patient a therapeutically effective amount of a compound of structural formula I and an HMG-CoA reductase inhibitor.

More particularly, this aspect of combination therapy concerns a method of treating a condition selected from the group consisting of hypercholesterolemia, atherosclerosis, low HDL levels, high LDL levels, hyperlipidemia, hypertriglyceridemia and dyslipidemia in a mammalian patient in need of such treatment wherein the HMG-CoA reductase inhibitor is a statin selected from the group consisting of lovastatin, simvastatin, pravastatin, cerivastatin, fluvastatin, atorvastatin, and rosuvastatin.

In another aspect of the invention, a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions is disclosed comprising administering to a mammalian patient in need of such treatment a therapeutically effective amount of a compound of structural formula I and an HMG-CoA reductase inhibitor.

In another aspect of the invention, a method for delaying the onset or reducing the risk of developing atherosclerosis in a human patient in need of such treatment is disclosed comprising administering to said patient an effective amount of a compound of structural formula I and an HMG-CoA reductase inhibitor.

More particularly, a method for delaying the onset or reducing the risk of developing atherosclerosis in a human patient in need of such treatment is disclosed, wherein the HMG-CoA reductase inhibitor is a statin selected from the group consisting of: lovastatin, simvastatin, pravastatin, cerivastatin, fluvastatin, atorvastatin, and rosuvastatin.

In another aspect of the invention, a method for delaying the onset or reducing the risk of developing atherosclerosis in a human patient in need of such treatment is disclosed, wherein the HMG-CoA reductase inhibitor is a statin and further comprising administering a cholesterol absorption inhibitor.

More particularly, in another aspect of the invention, a method for delaying the onset or reducing the risk of developing atherosclerosis in a human patient in need of such treatment is disclosed, wherein the HMG-CoA reductase inhibitor is a statin and the cholesterol absorption inhibitor is ezetimibe.

In another aspect of the invention, a pharmaceutical composition is disclosed which comprises:
(1) a compound of structural formula I;
(2) a compound selected from the group consisting of:

(a) dipeptidyl peptidase IV (DPP-IV) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (NN-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), melanin-concentrating hormone (MCH) receptor antagonists, and microsomal triglyceride transfer protein (MTP) inhibitors;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib; and

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors; and

(u) AMPK activators; and
(3) a pharmaceutically acceptable carrier.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require inhibition of stearoyl-CoA delta-9 desaturase enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing diabetes mellitus and/or hyperglycemia or hypertriglyceridemia or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Preparation of Compounds of the Invention:

The compounds of structural formula (I) can be prepared according to the procedures of the following Schemes and Examples, using appropriate materials and are further exemplified by the following specific examples. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted. Mass spectra (MS) were measured by electrospray ion-mass spectroscopy (ESMS).

### LIST OF ABBREVIATIONS

Alk=alkyl
APCI=atmospheric pressure chemical ionization
Ar=aryl
Boc=tert-butoxycarbonyl
br=broad
Cbz=benzyloxycarbonyl
CH₂Cl₂=dichloromethane
CH₂N₂=diazomethane
d=doublet
DBU=1,8-diazabicyclo[5.4.0]undec-7-ene
DCC=N,N′-dicyclohexylcarbodiimide
DCM=dichloromethane
DEAD=diethyl azodicarboxylate
Deoxofluor®=bis(2-methoxyethyl)aminosulfur trifluoride
DIPEA=N,N-diisopropylethylamine
DMF=N,N-dimethylformamide
DMSO=dimethyl sulfoxide
ESI=electrospray ionization
EtOAc=ethyl acetate
HATU=O-(7-azabenzotriazol-1-yl)-N,N,N,N′-tetramethyluronium hexafluorophosphate
HOAc=acetic acid
HOBt=1-hydroxybenzotriazole hydrate
KOH=potassium hydroxide
LC-MS=liquid chromatography-mass spectroscopy
LiOH=lithium hydroxide
m=multiplet
m-CPBA=3-chloroperoxybenzoic acid
MeOH=methyl alcohol
MgSO₄=magnesium sulfate
MMPP=magnesium monoperoxyphthalate
MS=mass spectroscopy
NaHMDS=sodium bis(trimethylsilyl)amide
NaOH=sodium hydroxide
Na₂SO₄=sodium sulfate
NH₄OAc=ammonium acetate
NMP=N-methylpyrrolidinone
NMR=nuclear magnetic resonance spectroscopy
PG=protecting group
rt=room temperature
s=singlet
t=triplet
THF=tetrahydrofuran
TFA=trifluoroacetic acid
TFAA=trifluoroacetic anhydride
TLC=thin-layer chromatography
TsCl=p-toluenesulfonyl chloride
p-TsOH=p-toluenesulfonic acid

### Method A:

An appropriately substituted heteroaryl halide 1 is reacted with an appropriately substituted cyclic amine 2 in the presence of a base such as 1,8-diazabicyclo[5.4.0]undec-7-ene (DBU), triethylamine or an alkali metal (K, Na, Cs) carbonate in a solvent such as N,N-dimethylformamide (DMF), ethanol, 2-methoxyethanol, and aqueous mixtures thereof at a temperature range of about room temperature to about refluxing temperature. Extractive work up and purification by flash column chromatography or precipitation of the product by the addition of saturated sodium bicarbonate solution or water gives desired condensed product 3.

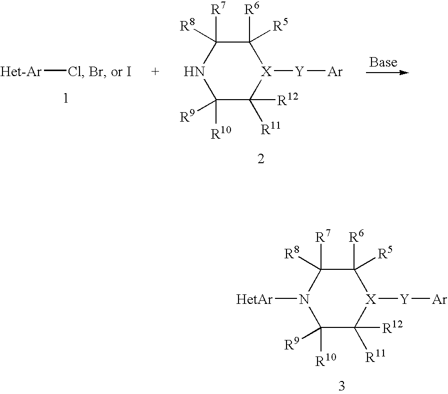

### Method B:

An appropriately substituted diaminoheteroaryl halide 4 is reacted with an appropriately substituted cyclic amine 2 in the presence of a base such as 1,8-diazabicyclo[5.4.0]undec-7-ene (DBU), triethylamine or an alkali metal (K, Na, Cs) carbonate in a solvent such as N,N-dimethylformamide (DMF), ethanol, 2-methoxyethanol, and aqueous mixtures thereof at a temperature range of about room temperature to about refluxing temperature. Extractive work up and purification by column chromatography or precipitation of the product by the addition of saturated sodium bicarbonate solution or water gives desired condensed product 5. Reaction of the diamino compound 5 with tert-butyl nitrite in a solvent such as dioxane under refluxing condition gives the desired triazole 6. Alkylation with a halo ester such as ethyl bromoacetate in the presence of a base such as NaH, Cs₂CO₃ or KOt-Bu in a solvent such as DMF usually gives a mixture of 7, 8 and 9, which can be separated by chromatography. Hydrolysis of the ester groups in 7, 8 and 9 with an alkaline base, such as sodium hydroxide, in a solvent such as THF and an alcoholic solvent such as MeOH at a temperature range of room temperature to refluxing temperature gives the carboxylic acids 10, 11 and 12, respectively.

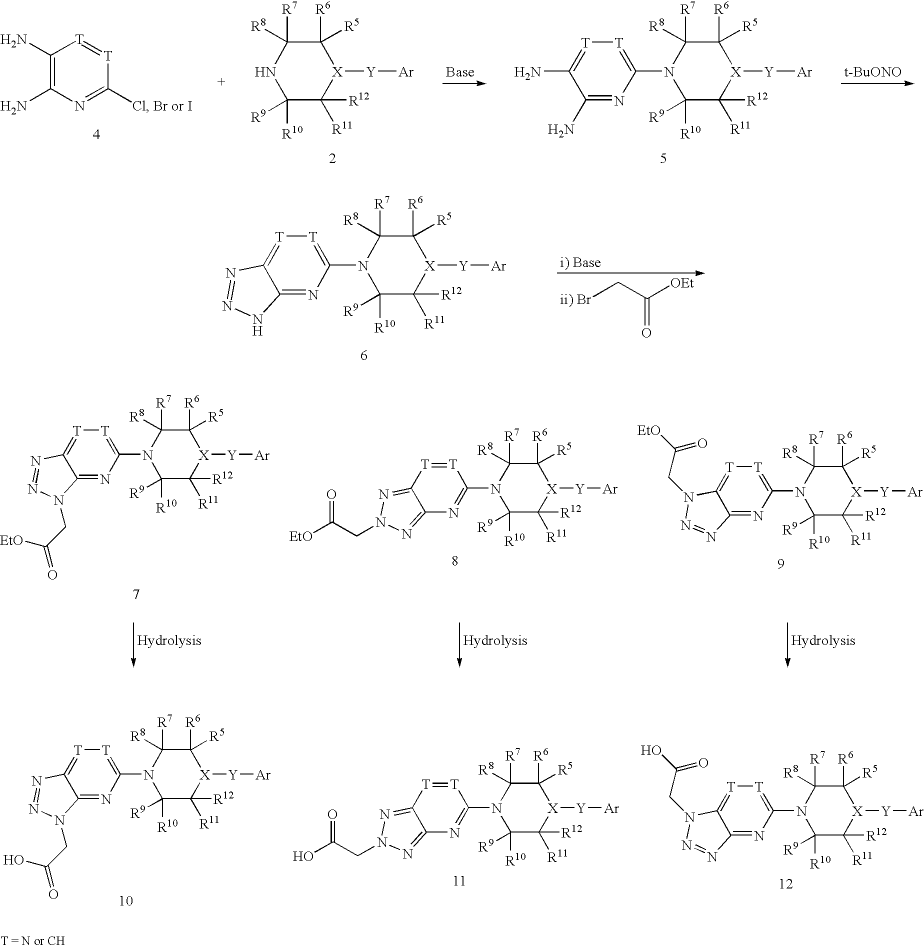

### Method C:

An appropriately substituted heteroaryl halide 13 is reacted with an appropriately substituted cyclic amine 2 in the presence of a base such as 1,8-diazabicyclo[5.4.0]undec-7-ene (DBU), triethylamine or an alkali metal (K, Na, Cs) carbonate in a solvent such as N,N-dimethylformamide (DMF), ethanol, 2-methoxyethanol, and aqueous mixtures thereof at a temperature range of about room temperature to about refluxing temperature. Extractive work up and purification by column chromatography or precipitation of the product by the addition of saturated sodium bicarbonate solution or water gives desired condensed product 14. Treatment of heteroarylamines 14 with t-butyl nitrite and anhydrous copper(II) halide in a solvent such as CH₃CN gives the desired heteroaryl halide 15. Displacement of the halide 15 with an appropriately substituted acetic acid ester in the presence of a base such as 1,8-diazabicyclo[5.4.0]undec-7-ene (DBU), triethylamine, sodium hydride or an alkali metal (K, Na, Cs) carbonate in a solvent such as N,N-dimethylformamide (DMF), THF or ethanol gives the condensed product 16. Hydrolysis of the ester group in 16 with an alkaline base such as sodium hydroxide in a solvent such as THF and an alcoholic solvent such as MeOH at a temperature range of room temperature to refluxing gives the carboxylic acids 17.

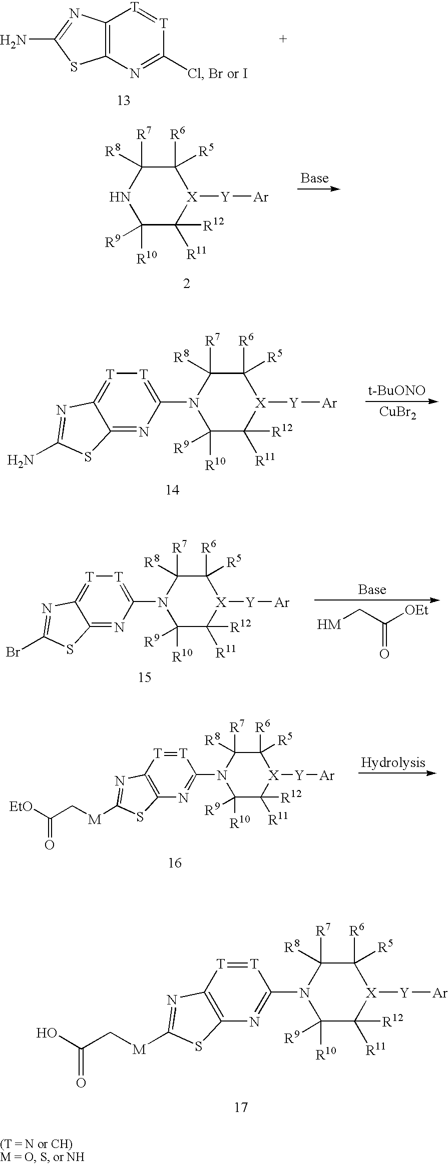

### Preparation of Intermediates:

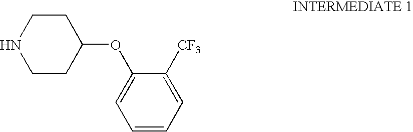

### 4-[2-(Trifluoromethyl)phenoxy]piperidine

To a solution of tert-butyl 4-hydroxypiperidine-1-carboxylate, 2-hydroxy-benzotrifluoride (1.1 equiv) and triphenylphosphine (1.2 equiv) in THF was added diethyl azodicarboxylate (1.2 equiv) dropwise at 0° C. The mixture was then warmed to room temperature and stirred for 14 h. The mixture was concentrated and diluted with Et₂O, washed with 1 N NaOH and water and then dried over Na₂SO₄. The mixture was concentrated and diluted with Et₂O/hexanes (35:65). The solid was filtered and the filtrate was concentrated. The residue was purified by column chromatography on silica gel (eluting with 35% Et₂O/hexanes) to give tert-butyl 4-[2-(trifluoromethyl)phenoxy]-piperidine-1-carboxylate as a solid.

Trifluoroacetic acid (2.75 equiv) was added to a solution of tert-butyl 4-[2-(trifluoromethyl)phenoxy]piperidine-1-carboxylate in CH₂Cl₂ (0.5 M). The mixture was stirred at room temperature for 16 h. The solvent was evaporated. The residue was diluted with EtOAc, washed with 2 N NaOH, brine, dried over Na₂SO₄, and evaporated to give the title compound as an oil.

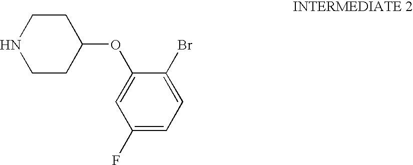

### 4-(2-Bromo-5-fluorophenoxy)piperidine

To a solution of tert-butyl 4-hydroxypiperidine-1-carboxylate in CH₂Cl₂ (0.5 M) was added methanesulfonyl chloride (1.2 equiv) and Et₃N (1.7 equiv) at 0° C. The mixture was further stirred for 3 h and filtered. The filtrate was evaporated in vacuo to give tert-butyl 4-[(methylsulfonyl)oxy]-piperidine-1-carboxylate. ¹H NMR (400 MHz, CDCl₃): δ 4.84-4.91 (m, 1H), 3.64-3.75 (m, 2H), 3.24-3.35 (m, 2H), 3.04 (s, 3H), 1.91-2.02 (m, 2H), 1.76-1.87 (m, 2H), 1.48 (s, 9H). MS: m/z 280 (MH⁺).

A solution of tert-butyl 4-[(methylsulfonyl)oxy]piperidine-1-carboxylate in DMF (1.0 M) was added 2-bromo-5-fluorophenol (1.2 equiv) and Cs₂CO₃ (2.0 equiv). The reaction mixture was heated at 70° C. overnight. The solvent was evaporated in vacuo, and the residue was purified by column chromatography on silica gel to give tert-butyl 4-(2-bromo-5-fluorophenoxy)piperidine-1-carboxylate. The product was used directly in next step without purification.

A solution of tert-butyl 4-(2-bromo-5-fluorophenoxy)piperidine-1-carboxylate in EtOH (4.5 M) was added dropwise 5 N HCl in EtOH solution (1.3 equiv). The reaction mixture was stirred at room temperature for 12 h. The solvent was evaporated in vacuo, and Et₂O was added to the residue. The resulting precipitate was washed with Et₂O to afford the title compound in the form of its hydrochloride salt. ¹H NMR (300 MHz, D₂O): δ 7.44-7.49 (m, 1H), 6.83-6.88 (m, 1H), 6.50-6.67 (m, 1H), 4.67-4.73 (m, 1H), 3.30-3.39 (m, 2H), 3.13-3.23 (m, 2H), 2.03-2.08 (m, 4H).

The salt was neutralized with aqueous 1 N NaOH, extracted with EtOAc, washed with brine, dried over Na₂SO₄, filtered and concentrated under reduced pressure. The title compound was used without further purification.

The following examples are provided to illustrate the invention and are not to be construed as limiting the invention in scope in any manner.

### Example 1

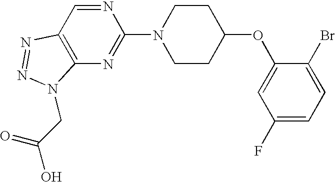

### {5-[4-(2-Bromo-5-fluorophenoxy)piperidin-1-yl]-3H-[1,2,3]triazolo[4,5-d]pyrimidin-3-yl}acetic acid

### Step 1: 2-[4-(2-Bromo-5-fluorophenoxy)piperidin-1-yl]pyrimidine-4,5-diamine

A mixture of 2-chloropyrimidine-4,5-diamine (3 g, 20.75 mmol), 4-(2-bromophenoxy)piperidine (6.83 g, 24.90 mmol) and Hunig's base (7.25 ml, 41.5 mmol) in 2-methoxyethanol (33.2 mL) and water (8.30 mL) was heated at 130° C. for 6 d. The solvent was evaporated, the residue diluted with water (25 mL) and extracted three times with EtOAc (25 mL). The combined organic fractions were dried over Na₂SO₄ and the solvent evaporated. Purification by Combiflash (SiO₂-120 g, gradient elution of 5% MeOH/EtOAc over 25 min) afforded the title product as a solid.

¹H NMR (500 MHz, acetone-d₆): δ 7.57 (dd, 1H), 7.53 (s, 1H), 7.04 (dd, 1H), 6.70 (td, 1H), 5.55 (s, 2H), 4.78-4.73 (m, 1H), 4.05-3.97 (m, 2H), 3.58-3.51 (m, 2H), 3.42 (s, 2H), 2.00-1.92 (m, 2H), 1.74-1.66 (m, 2H). MS (+ESI) m/z 382, 384 (MH⁺).

### Step 2: 5-[4-(2-Bromo-5-fluorophenoxy)piperidin-1-yl]-3H-[1,2,3]triazolo[4,5-d]-pyrimidine

To a mixture of 2-[4-(2-bromo-5-fluorophenoxy)piperidin-1-yl]pyrimidine-4,5-diamine (245 mg, 0.641 mmol) in dioxane (1.3 mL) was added tert-butyl nitrite (0.1 mL, 0.769 mmol). The mixture was heated at 70° C. for 8 h. The solvent was evaporated and the solid crystallized from CH₂Cl₂/hexanes, filtered and washed with hexanes to afford the title product as a solid.

¹H NMR (500 MHz, acetone-d₆): δ 9.15 (s, 1H), 7.59 (dd, 1H), 7.09 (dd, 1H), 6.73 (td, 1H), 4.94-4.89 (m, 1H), 4.21-4.01 (m, 4H), 2.11-2.05 (m, 2H), 1.87 (dtd, 2H). MS (+ESI) m/z 393, 395 (MH⁺).

### Step 3: Ethyl {5-[4-(2-bromo-5-fluorophenoxy)piperidin-1-yl]-3H-[1,2,3]triazolo[4,5-d]pyrimidin-3-yl}acetate

To a solution of 5-[4-(2-bromo-5-fluorophenoxy)piperidin-1-yl]-3H-[1,2,3]triazolo[4,5-d]-pyrimidine (500 mg, 1.272 mmol) in DMF (4.2 mL) was added sodium hydride (102 mg, 2.54 mmol). After 5 min, ethyl bromoacetate (0.2 mL, 1.907 mmol) was added and mixture heated at 80° C. After 30 min, the mixture was diluted with water (10 mL) and extracted three times with EtOAc (5 mL). The combined organic fractions were washed with water (10 mL) and dried over Na₂SO₄. The solvent was removed and purification by Combiflash (SiO₂-40 g, gradient elution of 5-40% EtOAc/hexanes over 30 min) afforded the title compound as the first eluted regioisomer.

¹H NMR (500 MHz, acetone-d₆): δ 9.13 (s, 1H), 7.58 (dd, 1H), 7.07 (dd, 1H), 6.72 (td, 1H), 5.37 (s, 2H), 4.92-4.88 (m, 1H), 4.22 (q, 2H), 4.15 (br s, 2H), 4.06 (br d, 2H), 2.13-2.03 (m, 2H), 1.90-1.83 (m, 2H), 1.24 (t, 3H). MS (+ESI): m/z 479, 481 (MH⁺).

The second eluted regioisomer was ethyl {5-[4-(2-bromo-5-fluorophenoxy)piperidin-1-yl]-2H-[1,2,3]triazolo[4,5-d]pyrimidin-2-yl}acetate:

¹H NMR (500 MHz, acetone-d₆): δ 9.32 (d, 1H), 7.59 (dd, 1H), 7.09 (dd, 1H), 6.72 (td, 1H), 5.56 (s, 2H), 4.94-4.89 (m, 1H), 4.33-4.12 (m, 4H), 4.14-4.00 (m, 2H), 2.12-2.05 (m, 2H), 1.91-1.84 (m, 2H), 1.28-1.21 (m, 3H). MS (+ESI): m/z 479, 481 (MH⁺).

The third eluted regioisomer was ethyl {5-[4-(2-bromo-5-fluorophenoxy)piperidin-1-yl]-3H-[1,2,3]triazolo[4,5-d]pyrimidin-3-yl}acetate:

¹H NMR (500 MHz, acetone-d₆): δ 9.21 (s, 1H), 7.59 (dd, 1H), 7.10 (dd, 1H), 6.72 (td, 1H), 5.68 (s, 2H), 4.93-4.88 (m, 1H), 4.27-4.14 (m, 4H), 4.07-3.97 (m, 2H), 2.13-2.08 (m, 2H), 1.91-1.83 (m, 2H), 1.35-1.21 (m, 3H). MS (+ESI): m/z 479, 481 (MH⁺).

### Step 4: {5-[4-(2-Bromo-5-fluorophenoxy)piperidin-1-yl]-3H-[1,2,3]triazolo[4,5-d]pyrimidin-3-yl}acetic acid

To a solution of the ethyl {5-[4-(2-bromo-5-fluorophenoxy)piperidin-1-yl]-3H-[1,2,3]triazolo[4,5-d]pyrimidin-3-yl}acetate (90 mg, 0.188 mmol) in THF (0.6 mL) and MeOH (0.3 mL) was added 1N NaOH (0.38 mL, 0.38 mmol). The mixture was stirred at RT for 10 min. The THF and MeOH were removed evaporated under diminished pressure and the mixture washed twice with Et₂O (2 mL). After adjusting the pH to about 1 with 1N HCl, the mixture was extracted three times with EtOAc (2 mL). The combined organic fractions were dried over Na₂SO₄ and the solvent was evaporated to afford the title compound as a solid.

¹H NMR (500 MHz, acetone-d₆): δ 9.14 (s, 1H), 7.59 (dd, 1H), 7.10 (dd, 1H), 6.73 (td, 1H), 5.38 (s, 2H), 4.94-4.89 (m, 1H), 4.18 (br s, 2H), 4.08 (br s, 2H), 2.12-2.06 (m, 2H), 1.91-1.84 (m, 2H). MS (+ESI): m/z 451, 453 (MH⁺).

### Example 2

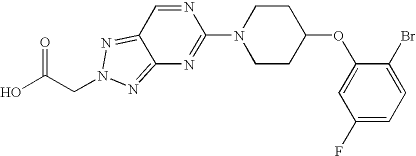

### {5-[4-(2-Bromo-5-fluorophenoxy)piperidin-1-yl]-2H-[1,2,3]triazolo[4,5-d]pyrimidin-2-yl}acetic acid

The title compound was prepared in the same manner as described in Example 1, Step 4, starting with the second-eluted regioisomer, ethyl {5-[4-(2-bromo-5-fluorophenoxy)piperidin-1-yl]-2H-[1,2,3]triazolo[4,5-d]pyrimidin-2-yl}acetate, obtained in Example 1, Step 3.

¹H NMR (500 MHz, acetone-d₆): δ 9.32 (s, 1H), 7.59 (dd, 1H), 7.10 (dd, 1H), 6.73 (td, 1H), 5.57 (s, 2H), 4.94-4.90 (m, 1H), 4.18 (d, 2H), 4.05 (br s, 2H), 2.11-2.05 (m, 2H), 1.92-1.84 (m, 2H). MS (+ESI): m/z 451, 453 (MH⁺).

### Example 3

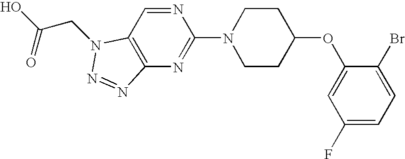

### {5-[4-(2-Bromo-5-fluorophenoxy)piperidin-1-yl]-3H-[1,2,3]triazolo[4,5-d]pyrimidin-1-yl}acetic acid

The title compound was prepared in the same manner as described in Example 1, Step 4, starting with the third-eluted regioisomer, ethyl {5-[4-(2-bromo-5-fluorophenoxy)piperidin-1-yl]-3H-[1,2,3]triazolo[4,5-d]pyrimidin-3-yl}acetate, obtained in Example 1, step 3.

¹H NMR (500 MHz, acetone-d₆): δ 9.24 (s, 1H), 7.59 (dd, 1H), 7.10 (dd, 1H), 6.73 (td, 1H), 5.68 (s, 2H), 4.92 (t, 1H), 4.22-4.15 (m, 2H), 4.03-3.97 (m, 2H), 2.12-2.06 (m, 2H), 1.91-1.84 (m, 2H). MS (+ESI): m/z 451, 453 (MH⁺).

### Example 4

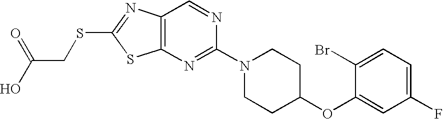

### ({5-[4-(2-Bromo-5-fluorophenoxy)piperidin-1-yl][1,3]thiazolo[5,4-d]pyrimidin-2-yl}thio)acetic acid

### Step 1: 2-Chloro[1,3]thiazolo[5,4-d]pyrimidin-2-amine

To a solution of 2-chloro-5-nitropyrimidin-4-yl thiocyanate (1.1 g, 5.08 mmol) [prepared as described in literature procedure Takahshi, T; Naito, T; Inoue, S Chem. Pharm. Bull. 1958; 6, 334-338], in AcOH (10.16 mL) was added iron (0.851 g, 15.24 mmol) and the mixture heated at 60° C. After 1 h, the mixture was filtered and the solvent evaporated. The residue was diluted with water (10 mL) and extracted three times with EtOAc (25 mL). The combined organic fractions were washed with 1N NaOH (50 mL) and dried over Na₂SO₄. The solvent was evaporated and the solid triturated with Et₂O to afford the title compound. MS (+ESI): m/z 187 (MH⁺).

### Step 2: 5-[4-(2-Bromo-5-fluorophenoxy)piperidin-1-yl][1,3]thiazolo[5,4-d]pyrimidin-2-amine

A mixture of 2-chloro[1,3]thiazolo[5,4-d]pyrimidin-2-amine (300 mg, 1.608 mmol), 4-(2-bromophenoxy)piperidine (529 mg, 1.929 mmol) and triethylamine (336 μL, 2.411 mmol) in DMF (3.2 mL) was heated at 120° C. for 3 h. The mixture was diluted with water (5 mL) and extracted three times with EtOAc (3 mL). The combined organic fractions were washed with water (3 mL) and dried over Na₂SO₄. The solvent was evaporated and the product recrystallized from CH₂Cl₂/hexanes, filtered and washed with hexanes to afford the title compound as a solid.

¹H NMR (500 MHz, acetone-d₆): δ 8.27 (s, 1H), 7.58 (dd, 1H), 7.08 (dd, 1H), 6.79 (d, 1H), 6.72 (td, 1H), 4.88-4.84 (m, 1H), 4.10-4.03 (m, 2H), 3.82 (ddd, 2H), 2.06-2.04 (m, 2H), 1.85-1.77 (m, 2H). MS (+ESI): m/z 424, 426 (MH⁺).

### Step 3: 2-Bromo-5-[4-(2-bromo-5-fluorophenoxy)piperidin-1-yl][1,3]thiazolo[5,4-d]pyrimidine

To a solution of 5-[4-(2-bromo-5-fluorophenoxy)piperidin-1-yl][1,3]thiazolo[5,4-d]pyrimidin-2-amine (247 mg, 0.582 mmol), in acetonitrile (7 mL) was added copper (II) bromide (195 mg, 0.873 mmol). After 5 min, tert-butyl nitrite (0.138 mL, 1.164 mmol) was added. The reaction was stirred at RT for 30 min. The solvent was evaporated under reduced pressure and the residue was diluted with water (20 mL) and EtOAc (20 mL). The mixture was filtered through celite and the aqueous layer extracted three times with EtOAc (75 mL). The combined organic fractions were washed twice with water (100 mL), dried (MgSO₄), filtered and the solvent was evaporated under reduced pressure. The residue was purified by column chromatography on silica gel eluting with 10% EtOAc/Hexanes to give the title compound as a white foam.

¹H NMR (500 MHz, acetone-d₆): δ 8.84 (s, 1H), 7.59 (dd, 1H), 7.10 (dd, 1H), 6.73 (td, 1H), 4.94-4.90 (m, 1H), 4.14-4.07 (m, 2H), 4.04-3.98 (m, 2H), 2.12-2.06 (m, 2H), 1.92-1.84 (m, 2H). MS (+ESI): m/z 489 (MH⁺).

### Step 4: Ethyl ({5-[4-(2-bromo-5-fluorophenoxy)piperidin-1-yl][1,3]thiazolo[5,4-d]pyrimidin-2-yl}thio)acetate

To a solution of 2-bromo-5-[4-(2-bromo-5-fluorophenoxy)piperidin-1-yl][1,3]thiazolo[5,4-d]pyrimidine (100 mg, 0.205 mmol) and Et₃N (57 μL, 0.409 mmol) in EtOH (2 mL) was added the ethyl-2-mercaptoacetate (26.9 μL, 0.246 mmol). The reaction was stirred at RT overnight. The precipitate was filtered and washed with EtOH (about 1 mL) and hexanes (about 6 mL). The title compound was obtained as a white solid.

¹H NMR (400 MHz, acetone-d₆): δ 8.68 (s, 1H), 7.58 (dd, 1H), 7.07 (dd, 1H), 6.72 (td, 1H), 4.92-4.85 (m, 1H), 4.22 (s, 2H), 4.22-4.13 (m, 2H), 4.15-4.06 (m, 2H), 4.00-3.92 (m, 2H), 2.08-2.04 (m, 2H), 1.90-1.80 (m, 2H), 1.24 (t, 3H). MS (+ESI): m/z 527, 529 (MH⁺).

### Step 5: ({5-[4-(2-Bromo-5-fluorophenoxy)piperidin-1-yl][1,3]thiazolo[5,4-d]pyrimidin-2-yl}thio)acetic acid

To a solution of ethyl ({5-[4-(2-bromo-5-fluorophenoxy)piperidin-1-yl][1,3]thiazolo[5,4-d]pyrimidin-2-yl}thio)acetate (35 mg, 0.066 mmol) in THF (0.88 mL), water (0.44 mL) and MeOH (0.44 mL) was added solution of 10 N NaOH (13.27 μL, 0.133 mmol). The reaction mixture was stirred at RT for 15 min. The solvents were evaporated and the pH was adjusted to pH 1 using 1 N HCl. The aqueous media was extracted three times with EtOAc (40 ml). The combined organic fractions were dried (MgSO₄), filtered and the solvent was evaporated under reduced pressure. The title compound was obtained as a white solid.

¹H NMR (500 MHz, acetone-d₆): δ 8.71 (s, 1H), 7.59 (dd, 1H), 7.09 (dd, 1H), 6.73 (td, 1H), 4.92-4.89 (m, 1H), 4.26 (s, 2H), 4.15-4.09 (m, 2H), 3.99-3.95 (m, 2H), 2.08-2.05 (m, 2H), 1.88-1.84 (m, 2H). MS (+ESI): m/z 499, 500 (MH⁺).

### Example 5

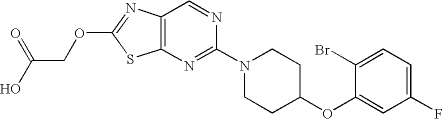

### ({5-[4-(2-Bromo-5-fluorophenoxy)piperidin-1-yl][1,3]thiazolo[5,4-d]pyrimidin-2-yl}oxy)acetic acid

### Step 1: Ethyl({5-[4-(2-bromo-5-fluorophenoxy)piperidin-1-yl][1,3]thiazolo[5,4-d]pyrimidin-2-yl}oxy)acetate

To a solution of ethyl glycolate (10.66 μL, 0.113 mmol) in DMF (0.50 ml) was added NaH (4.51 mg, 0.113 mmol). After 10 min, a solution of 2-bromo-5-[4-(2-bromo-5-fluorophenoxy)piperidin-1-yl][1,3]thiazolo[5,4-d]pyrimidine (50 mg, 0.102 mmol) [from example 4, step 3], in DMF (0.50 mL) was added and the reaction mixture was stirred at RT for 30 min. The mixture was acidified using 1N HCl and the aqueous phase was extracted three times with EtOAc (40 mL). The combined organic fractions were washed with water, dried (MgSO₄), filtered and the solvent was evaporated under reduced pressure. The residue was purified by column chromatography on silica gel eluting with 20% EtOAc/hexanes to give the title compound as an oil.

¹H NMR (400 MHz, acetone-d₆): δ 8.50 (s, 1H), 7.58 (dd, 1H), 7.07 (dd, 1H), 6.72 (td, 1H), 5.12 (s, 2H), 4.93-4.85 (m, 1H), 4.22 (q, 2H), 4.13-4.05 (m, 2H), 3.94-3.86 (m, 2H), 2.08 (m, 2H), 1.87-1.78 (m, 2H), 1.24 (t, 3H). MS (+ESI): m/z 510, 511 (MH⁺).

### Step 2: ({5-[4-(2-Bromo-5-fluorophenoxy)piperidin-1-yl][1,3]thiazolo[5,4-d]pyrimidin-2-yl}oxy)acetic acid

The title compound was prepared in the same manner as described in Example 4, step 5 from ethyl({5-[4-(2-bromo-5-fluorophenoxy)piperidin-1-yl][1,3]thiazolo[5,4-d]pyrimidin-2-yl}oxy)acetate and aqueous NaOH.

¹H NMR (500 MHz, acetone-d₆): δ 8.52 (s, 1H), 7.59 (dd, 1H), 7.09 (dd, 1H), 6.75-6.70 (m, 1H), 5.14 (s, 2H), 4.91-4.87 (m, 1H), 4.11-4.07 (m, 2H), 3.93-3.89 (m, 2H), 2.09-2.02 (m, 2H), 1.88-1.75 (m, 2H). MS (+ESI): m/z 483, 484 (MH⁺).

### Example 6

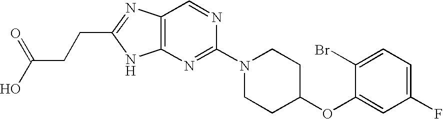

### 3-{2-[4-(2-Bromo-5-fluorophenoxy)piperidin-1-yl]-9H-purin-8-yl}propanoic acid}amino)-4-oxobutanoic acid

### Step 1: 4-({4-Amino-2-[4-(2-bromo-5-fluorophenoxy)piperidin-1-yl]pyrimidin-5-amino)-4-oxobutanoic acid

To a solution of 2-[4-(2-bromo-5-fluorophenoxy)piperidin-1-yl]pyrimidine-4,5-diamine (300 mg, 0.785 mmol) [from example 1, step 1], in THF (3.924 ml) was added NaH (94 mg, 2.355 mmol). After 10 min, succinic anhydride (0.095 mL, 1.177 mmol) was added and the reaction mixture was stirred at 80° C. for 1 h. The reaction was allowed to cool to RT and the solvent was evaporated under reduced pressure. The residue was diluted with water and acidified with acetic acid. The aqueous media was extracted three times with EtOAc (50 mL) and the combined organic fractions were dried (MgSO₄) and the solvent was evaporated under reduced pressure. The crude product was used in the next step without further purification. MS (+ESI): m/z 464, 466 (MH⁺).

### Step 2: 3-{2-[4-(2-Bromo-5-fluorophenoxy)piperidin-1-yl]-9H-purin-8-yl}propanoic acid}amino)-4-oxobutanoic acid

4-({4-Amino-2-[4-(2-bromo-5-fluorophenoxy)piperidin-1-yl]pyrimidin-5-yl}amino)-4-oxobutanoic acid (375 mg, 0.778 mmol) was dissolved in AcOH (5 mL). The reaction mixture was stirred at 120° C. for 2 h. The solvent was removed under reduced pressure and the residue obtained was diluted with 1N HCl (100 mL) and extracted three times with CH₂Cl₂ (50 ml). The combined organic phases were dried (MgSO₄) and the solvent evaporated under reduced pressure. The residue was recrystallized from CH₂Cl₂/hexanes to afford the title compound as a brown solid.

¹H NMR (500 MHz, acetone-d₆): δ 7.64 (s, 1H), 7.58 (dd, 1H), 7.07 (dd, 1H), 6.71 (td, 1H), 6.01 (s, 2H), 4.85-4.82 (m, 1H), 4.05-4.00 (m, 2H), 3.83-3.77 (m, 2H), 2.78 (s, 2H), 2.01-1.97 (m, 2H), 1.78-1.75 (m, 2H). MS (+ESI): m/z 464, 466 (MH⁺).

### Example 7

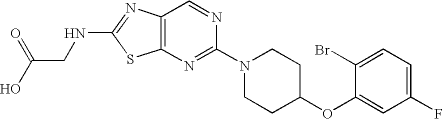

### ({5-[4-(2-Bromo-5-fluorophenoxy)piperidin-1-yl][1,3]thiazolo[5,4-d]pyrimidin-2-yl}amino)acetic acid

### Step 1: Methyl [(5-chloro[1,3]thiazolo[5,4-d]pyrimidin-2-yl)amino]acetate

2-Chloro[1,3]thiazolo[5,4-d]pyrimidin-2-amine (250 mg, 1.524 mmol) [from example 4, step 1] and ethyl isothiocyanatoacetate (2.0 mL, 16.12 mmol) were heated in a flask at 100° C. for 10 min. MeOH (5.0 mL) was then added and the temperature was adjusted to 75° C. After 18 h, the solvent was evaporated under reduce pressure. MeOH (about 2.0 mL) was added and the solid was filtered and washed with hexanes to afford the title compound as a solid.

¹H NMR (400 MHz, acetone-d₆): δ 8.54 (s, 1H), 8.20-8.16 (m, 1H), 4.39 (d, 2H), 3.73 (s, 3H). MS (+ESI): m/z 259 (MH⁺).

### Step 2: 2-Methoxyethyl({5-[4-(2-bromo-5-fluorophenoxy)piperidin-1-yl][1,3]thiazolo[5,4-d]pyrimidin-2-yl}amino)acetate

To a solution of methyl [(5-chloro[1,3]thiazolo[5,4-d]pyrimidin-2-yl)amino]acetate (140 mg, 0.541 mmol) and 4-(2-bromophenoxy)piperidine (178 mg, 0.649 mmol) in 2-methoxyethanol (2.7 mL) was added Et₃N (0.15 mL, 1.082 mmol). The reaction was heated overnight at 120° C. The reaction mixture was diluted with water and extracted three times with EtOAc (20 mL). The combined organic fractions were washed three times with water (20 mL), dried (MgSO₄), filtered and evaporated under reduce pressure. The residue was purified by column chromatography on silica gel, eluting with a gradient of from 40 to 60% EtOAc/Hexanes to give the title compound as a solid.

¹H NMR (500 MHz, acetone-d₆): δ 8.32 (s, 1H), 7.58 (dd, 1H), 7.50 (t, 1H), 7.08 (dd, 1H), 6.71 (dt, 1H), 4.86 (s, 1H), 4.29 (d, 2H), 4.25 (t, 2H), 4.10-4.04 (m, 2H), 3.83-3.80 (m, 2H), 3.57 (t, 2H), 3.29 (s, 3H), 2.03-2.00 (m, 2H), 1.84-1.79 (m, 2H). MS (+ESI): m/z 541, 542 (MH⁺).

### Step 3: ({5-[4-(2-Bromo-5-fluorophenoxy)piperidin-1-yl][1,3]thiazolo[5,4-d]pyrimidin-2-yl}amino)acetic acid

The title compound was prepared in the same manner as described in Example 4, step 5, from 2-methoxyethyl({5-[4-(2-bromo-5-fluorophenoxy)piperidin-1-yl][1,3]thiazolo[5,4-d]pyrimidin-2-yl}amino)acetate and aqueous NaOH.

¹H NMR (400 MHz, acetone-d₆): δ 8.32 (s, 1H), 7.58 (dd, 1H), 7.43 (t, 1H), 7.08 (dd, 1H), 6.71 (td, 1H), 4.88-4.83 (m, 1H), 4.27 (d, 2H), 4.12-4.04 (m, 2H), 3.87-3.79 (m, 2H), 2.05-2.00 (m, 2H), 1.85-1.76 (m, 2H). MS (+ESI): m/z 482, 483 (MH⁺).

### Example 8

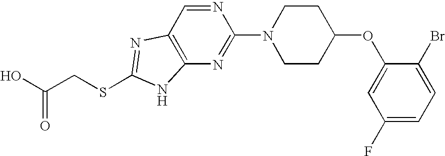

### ({2-[4-(2-Bromo-5-fluorophenoxy)piperidin-1-yl]-9H-purin-8-yl}thio)acetic acid

### Step 1: 2-[4-(2-Bromo-5-fluorophenoxy)piperidin-1-yl]-9H-purine-8-thiol

To a solution of 2-[4-(2-bromo-5-fluorophenoxy)piperidin-1-yl]pyrimidine-4,5-diamine (300 mg, 0.785 mmol) [from example 1, step 1], in EtOH (3.9 mL) was added carbon disulfide (0.056 mL, 0.942 mmol) followed by 1N sodium hydroxide (1.6 mL, 1.57 mmol). The mixture was heated at 90° C. for 2 h. The solvent was evaporated and the residue was diluted with 5% citric acid (5 mL) and Et₂O/hexanes 1:1 (5 mL). The mixture was filtered and washed with water followed by 1:1 Et₂O/hexanes. The solid was dried under high vacuum to afford the title product.

¹H NMR (500 MHz, acetone-d₆): δ 8.11 (s, 1H), 7.59 (dd, 1H), 7.07 (dd, 1H), 6.72 (d, 1H), 4.87 (d, 1H), 4.08-4.03 (m, 2H), 3.86-3.80 (m, 2H), 2.03 (d, 2H), 1.83-1.78 (m, 2H). MS (+ESI): m/z 424, 426 (MH⁺).

### Step 2: Ethyl({2-[4-(2-bromo-5-fluorophenoxy)piperidin-1-yl]-9H-purin-8-yl}thio)acetate

To a mixture of 2-[4-(2-bromo-5-fluorophenoxy)piperidin-1-yl]-9H-purine-8-thiol (120 mg, 0.283 mmol) and ethyl bromoacetate (0.04 mL, 0.339 mmol) in THF (0.94 mL) was added triethylamine (0.08 mL, 0.566 mmol). The mixture was stirred at RT for 2 h. The solvent was evaporated, and the residue was slurried with 1:1 Et₂O/hexanes (2 mL) and water (2 mL). The solid was filtered and washed with 1:1 Et₂O/hexanes. The product was recrystallized from CH₂Cl₂/hexanes, filtered and washed with hexanes to afford the title product as a solid. MS (+ESI): m/z 510, 512 (MH⁺).

### Step 3: ({2-[4-(2-Bromo-5-fluorophenoxy)piperidin-1-yl]-9H-purin-8-yl}thio)acetic acid

The title compound was prepared in the same manner as described in Example 4, step 5 from ethyl ({2-[4-(2-bromo-5-fluorophenoxy)piperidin-1-yl]-9H-purin-8-yl}thio)acetate and aqueous NaOH.

¹H NMR (500 MHz, acetone-d₆): δ 8.52 (s, 1H), 7.63-7.59 (m, 1H), 7.10 (d, 1H), 6.74 (t, 1H), 4.89 (s, 1H), 4.18-4.11 (m, 4H), 3.90-3.83 (m, 2H), 2.11-2.073 (m, 2H), 1.87-1.79 (m, 2H). MS (+ESI): m/z 482, 484 (MH⁺).

### Example 9

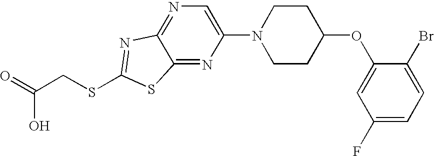

### ({6-[4-(2-Bromo-5-fluorophenoxy)piperidin-1-yl][1,3]thiazolo[4,5-b]pyrazin-2-yl}thio)acetic acid

### Step 1: 2,6-Dibromo[1,3]thiazolo[4,5-b]pyrazine

To a solution of 6-bromo[1,3]thiazolo[4,5-b]pyrazin-2-amine (300 mg, 1.298 mmol) [prepared as described by Koren, B.; Stanovinik, B.; Tisler, M. Heterocycles 1987, 3, 689] in acetonitrile (13.0 ml) was added copper (II) bromide (435 mg, 1.947 mmol). After 5 min, tert-butyl nitrite (0.308 ml, 2.59 mmol) was added. The reaction was stirred at RT for 18 h. The solvent was evaporated under reduced pressure and the residue was diluted with water (20 mL) and EtOAc (20 mL). The mixture was filtered through celite and the aqueous layer extracted three times with EtOAc. The combined organic extracts were washed twice with water (100 mL), dried (MgSO₄), filtered and the filtrate evaporated under reduced pressure to afford the title compound as a solid.

¹H NMR (500 MHz, acetone-d₆): δ 8.87 (s, 1H). MS (+ESI) m/z 292, 295 (MH⁺).

### Step 2: Ethyl [(6-bromo[1,3]thiazolo[4,5-b]pyrazin-2-yl)thio]acetate

To a solution of 2,6-dibromo[1,3]thiazolo[4,5-b]pyrazine (50 mg, 0.170 mmol) in EtOH (0.848 mL) was added Et₃N (47.3 μL, 0.339 mmol) and ethyl 2-mercaptoacetate (20.37 μL, 0.186 mmol). The reaction mixture was stirred at RT for 30 min. The solvent was evaporated under reduced pressure and the residue was diluted with water (10 mL) and EtOAc (5 mL). The aqueous layer was extracted three times with EtOAc (15 mL) and the combined organic fractions were dried (MgSO₄), filtered and evaporated under reduced pressure to afford the title compound as a solid.

¹H NMR (500 MHz, acetone-d₆): δ 8.73 (s, 1H), 4.38 (s, 2H), 4.21 (q, 2H), 1.25 (t, 3H). MS (+ESI) m/z 333 (MH⁺).

### Step 3: Ethyl({6-[4-(2-bromo-5-fluorophenoxy)piperidin-1-yl][1,3]thiazolo[4,5-b]pyrazin-2-yl}thio)acetate

Ethyl [(6-bromo[1,3]thiazolo[4,5-b]pyrazin-2-yl)thio]acetate (63 mg, 0.189 mmol) and 4-(2-bromo-5-fluorophenoxy)piperidine (62 mg, 0.226 mmol) were dissolved in DMF (0.628 mL). Et₃N (52.5 μL, 0.377 mmol) was added and the reaction mixture was stirred at 100° C. for 3 h. The reaction mixture was diluted with water (5 mL) and extracted three times with EtOAc (5 mL). The combined organic phases were washed twice with water (5 mL), dried (MgSO₄), filtered and evaporated under reduced pressure. The residue was purified by Combiflash (SiO₂-12 g, gradient elution of 0-30% EtOAc/Hexanes over 30 min). The title compound was afforded as a solid.

¹H NMR (500 MHz, acetone-d₆): δ 8.33 (s, 1H), 7.59 (dd, 1H), 7.09 (dd, 1H), 6.72 (td, 1H), 4.95-4.88 (m, 1H), 4.26 (s, 2H), 4.19 (q, 2H), 4.01-3.93 (m, 2H), 3.83 (ddd, 2H), 2.18-2.08 (m, 2H), 1.98-1.88 (m, 2H), 1.24 (t, 3H). MS (+ESI) m/z 528 (MH⁺).

### Step 4: ({6-[4-(2-Bromo-5-fluorophenoxy)piperidin-1-yl][1,3]thiazolo[4,5-b]pyrazin-2-yl}thio)acetic acid

The title compound was prepared in the same manner as described in Step 5 of Example 4 from ethyl({6-[4-(2-bromo-5-fluorophenoxy)piperidin-1-yl][1,3]thiazolo[4,5-b]pyrazin-2-yl}thio)acetate and aqueous NaOH.

¹H NMR (500 MHz, acetone-d₆): δ 8.34 (s, 1H), 7.59 (dd, 1H) 7.09 (dd, 1H), 6.73 (td, 1H), 4.92 (m, 1H), 4.28 (s, 2H), 3.97 (m, 2H), 3.84 (m, 2H), 2.13 (m, 2H), 1.94 (m, 2H). MS (+ESI) m/z 499 (MH⁺).

### Example 10

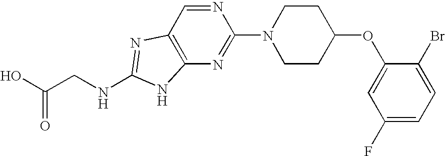

### N-(2-{4-[(2-bromo-5-fluorophenyl)oxy]piperidin-1-yl}-9H-purin-8-yl)glycine

### Step 1: Ethyl N-(2-{4-[(2-bromo-5-fluorophenyl)oxy]piperidin-1-yl}-9H-purin-8-yl)glycinate

A solution of 2-[4-(2-bromo-5-fluorophenoxy)piperidin-1-yl]pyrimidine-4,5-diamine (200 mg, 0.523 mmol) and ethyl isothiocyanatoacetate (78 μL, 0.628 mmol) in THF (1.7 mL) was heated at 80° C. for 1 h. DCC (130 mg, 0.628 mmol) was added and the mixture was heated at 80° C. for 3 h. The solvent was evaporated and the crude product was purified by Combiflash chromatography (SiO₂-12 g, gradient elution of 0-5% MeOH/EtOAc over 25 min) to afford the title product.

¹H NMR (500 MHz, acetone-d₆): δ 8.09 (s, 1H), 7.64-7.57 (m, 1H), 7.09 (dd, 1H), 6.73 (td, 1H), 4.86-4.81 (m, 1H), 4.33-4.09 (m, 6H), 3.78-3.71 (m, 2H), 2.07-2.01 (m, 2H), 1.83-1.75 (m, 2H), 1.25 (td, 3H). MS (+ESI) m/z 493, 495 (MH⁺).

### Step 2: N-(2-{4-[(2-bromo-5-fluorophenyl)oxy]piperidin-1-yl}-9H-purin-8-yl)glycine

The title compound was prepared in the same manner as described in Example 4, step 5 from ethyl N-(2-{4-[(2-bromo-5-fluorophenyl)oxy]piperidin-1-yl}-9H-purin-8-yl)glycinate and aqueous NaOH.

¹H NMR (500 MHz, acetone-d₆): δ 8.09 (s, 1H), 7.62 (dd, 1H), 7.09 (dd, 1H), 6.77 (td, 1H), 4.85 (s, 1H), 4.26 (s, 2H), 4.18-4.11 (m, 2H), 3.79-3.72 (m, 2H), 2.05-1.99 (m, 2H), 1.85-1.75 (m, 2H). MS (+ESI) m/z 465, 467 (MH⁺).

### Example 11

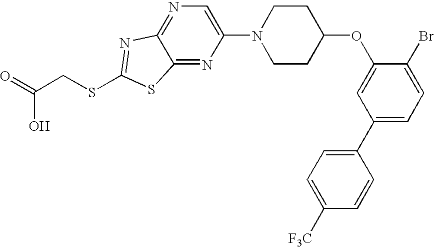

### {[6-(4-{[4-Bromo-4′-(trifluoromethyl)biphenyl-3-yl]oxy}piperidin-1-yl)[1,3]thiazolo[4,5-b]pyrazin-2-yl]thio}acetic acid

### Step 1: 4-Bromo-4′-(trifluoromethyl)biphenyl-3-ol

To a solution of 2-bromo-5-iodophenol (5.0 g, 16.75 mmol) and 4-(trifluoromethyl)phenyl boronic acid (4.2 g, 22.26 mmol) in toluene (25 mL) was added aqueous 2 M Na₂CO₃ (25 mL, 50.0 mmol), and Pd(Ph₃P)₄ (500 mg, 0.433 mmol). After the resulting heterogeneous mixture was purged with nitrogen, it was gently heated to 45° C. for 1.5 h with stirring under nitrogen atmosphere. After 1.5 h, extra amount of reagents (2 M Na₂CO₃ (5 mL, 10.00 mmol), Pd(Ph₃P)₄ (103 mg, 0.089 mmol), 4-(trifluoromethyl)phenyl boronic acid (810 mg, 4.26 mmol)) were added and heating to 45° C. was pursued for an additional 1 h. After cooling to room temperature, the reaction was poured into aqueous 1 N HCl (30 mL), extracted with EtOAc (15 ml) and washed with brine (20 mL). The organic layer was dried (Na₂SO₄), treated with active charcoal and filtered through a pad of celite. The solvents were removed under reduced pressure and the residue was purified by column chromatography on silica gel eluting with toluene. After evaporation of the solvents, the residue was heated with a heat gun under high vacuum to remove unreacted starting material 2-bromo-5-iodophenol and the title compound was obtained as a solid.

¹H NMR (400 MHz, acetone-d₆): δ 9.04 (s, 1H), 7.83 (d, 2H), 7.79 (d, 2H), 7.63 (d, 1H), 7.32 (d, 1H), 7.15 (dd, 1H). MS (+ESI): m/z 317, 315 (MH⁺).

### Step 2: Ethyl{[6-(4-{[4-bromo-4′-(trifluoromethyl)biphenyl-3-yl]oxy}piperidin-1-yl)[1,3]thiazolo[4,5-b]pyrazin-2-yl]thio}acetate

The title compound was prepared in the same manner as described in Step 3 of Example 9 from ethyl [(6-bromo[1,3]thiazolo[4,5-b]pyrazin-2-yl)thio]acetate and 4-bromo-4′-(trifluoromethyl)biphenyl-3-ol.

¹H NMR (500 MHz, acetone-d₆): δ 8.32 (s, 1H), 7.90 (d, 2H), 7.79 (d, 2H), 7.70 (d, 1H), 7.55 (d, 1H), 7.26 (dd, 1H), 5.10-5.06 (m, 1H), 4.26 (s, 2H), 4.18 (q, 2H), 4.03-3.94 (m, 2H), 3.87-3.80 (m, 2H), 2.19-2.12 (m, 2H), 2.02-1.94 (m, 2H), 1.24 (t, 3H), MS (+ESI): m/z 653, 655 (MH⁺).

### Step 3: {[6-(4-{[4-Bromo-4′-(trifluoromethyl)biphenyl-3-yl]oxy}piperidin-1-yl)[1,3]thiazolo[4,5-b]pyrazin-2-yl]thio}acetic acid

The title compound was prepared in the same manner as described in Step 5 of Example 4 from ethyl{[6-(4-{[4-bromo-4′-(trifluoromethyl)biphenyl-3-yl]oxy}piperidin-1-yl)[1,3]thiazolo[4,5-b]pyrazin-2-yl]thio}acetate and aqueous NaOH.

¹H NMR (500 MHz, acetone-d₆): δ 8.34 (s, 1H), 7.91 (d, 2H), 7.80 (d, 2H), 7.71 (d, 1H), 7.57 (d, 1H), 7.27 (dd, 1H), 5.10-5.08 (m, 1H), 4.29 (s, 2H), 4.02-3.96 (m, 2H), 3.87-3.83 (m, 2H), 2.18-2.13 (m, 2H), 1.93-1.91 (m, 2H). MS (+ESI) m/z 625, 627 (MH⁺).

### Example 12

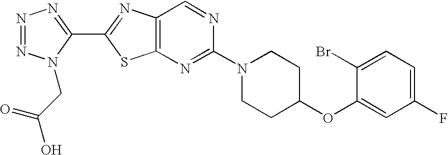

### (5-{5-[4-(2-Bromo-5-fluorophenoxy)piperidin-1-yl][1,3]thiazolo[5,4-d]pyrimidin-2-yl}-1H-tetrazol-1-yl)acetic acid

### Step 1: Methyl 5-chloro[1,3]thiazolo[5,4-d]pyrimidine-2-carboxylate

To a mixture of the 5-amino-2-chloropyrimidine-4-thiol (500 mg, 3.09 mmol) in THF (15.5 ml) was added sodium hydride (408 mg, 10.21 mmol) at 0° C. After 5 min, methyl oxalyl chloride (0.574 mL, 6.19 mmol) was added and the mixture allowed to warm to RT and then stirred for a further 30 min. The mixture was heated at 80° C. for 2 h. The solvent was evaporated under reduced pressure and the residue was diluted with Et₂O/Hexanes (1:1, 10 mL). The mixture was quenched with ice (about 10 g) and the solid precipitate was filtered, washed with water then hexanes. The solid was dried under high vacuum to afford the title compound as a solid.

¹H NMR (500 MHz, acetone-d₆): δ 9.54 (s, 1H), 4.12 (s, 3H). MS (+ESI) m/z 230 (MH⁺).

### Step 2: Methyl 5-[4-(2-bromo-5-fluorophenoxy)piperidin-1-yl][1,3]thiazolo[5,4-d]pyrimidine-2-carboxylate

The title compound was prepared in the same manner as described in Step 3 of Example 9 from methyl 5-chloro[1,3]thiazolo[5,4-d]pyrimidine-2-carboxylate and 4-(2-bromo-5-fluorophenoxy)piperidine (Intermediate 2).

¹H NMR (500 MHz, acetone-d₆): δ 9.08 (s, 1H), 7.60 (dd, 1H), 7.11 (dd, 1H), 6.75-6.70 (m, 1H), 4.96-4.92 (m, 1H), 4.19-4.14 (m, 2H), 4.11-4.06 (m, 2H), 4.00 (s, 3H), 2.11-2.06 (m, 2H), 1.93-1.88 (m, 2H). MS (+ESI) m/z 467, 469 (MH⁺).

### Step 3: 5-[4-(2-Bromo-5-fluorophenoxy)piperidin-1-yl][1,3]thiazolo[5,4-d]pyrimidine-2-carboxamide

To a solution of methyl 5-[4-(2-bromo-5-fluorophenoxy)piperidin-1-yl][1,3]thiazolo[5,4-d]pyrimidine-2-carboxylate (715 mg, 1.53 mmol) in THF (5 mL) was added MeOH (1 mL). Ammonia gas was bubbled into the solution for 5 min. The reaction was stirred at RT overnight and then heated at 50° C. for 6 h. The solvents were evaporated under reduced pressure. Trituration from DCM/Et₂O/hexanes afforded the title compound as a solid.

¹H NMR (500 MHz, acetone-d₆): δ 8.98 (s, 1H), 7.81-7.75 (m, 1H), 7.60 (dd, 1H), 7.22-7.20 (m, 1H), 7.10 (dd, 1H), 6.73 (td, 1H), 4.95-4.92 (m, 1H), 4.18-4.13 (m, 2H), 4.10-4.04 (m, 2H), 2.11-2.05 (m, 2H), 1.92-1.87 (m, 2H). MS (+ESI) m/z 452, 454 (MH⁺).

### Step 4: 5-[4-(2-Bromo-5-fluorophenoxy)piperidin-1-yl][1,3]thiazolo[5,4-d]pyrimidine-2-carbonitrile

To a solution of 5-[4-(2-bromo-5-fluorophenoxy)piperidin-1-yl][1,3]thiazolo[5,4-d]pyrimidine-2-carboxamide (250 mg, 0.55 mmol) and Et₃N (247 μL, 1.77 mmol) in THF (1.84 mL) was added TFAA (117 μL, 0.83 mmol) dropwise at 0° C. The reaction was allowed to warm to RT and stirred for 16 h. The solvent was evaporated under reduced pressure. The residue was diluted with EtOAc (15 mL) and NaHCO₃ (until basic pH), then extracted with EtOAc (2×15 mL). The combined organic fractions were dried (MgSO₄), filtered and evaporated under reduced pressure. Purification by Combiflash chromatography, (SiO₂-12 g, elution with 0-30% EtOAc/Hexanes over 20 min) afforded the title compound as a solid.

¹H NMR (400 MHz, acetone-d₆): δ 9.15 (s, 1H), 7.60 (dd, 1H), 7.10 (dd, 1H), 6.75 (dt, 1H), 4.97-4.94 (m, 1H), 4.20-4.12 (m, 4H), 2.10 (m, 2H), 1.92 (m, 2H). MS (+ESI) m/z 434, 436 (MH⁺).

### Step 5: 5-[4-(2-Bromo-5-fluorophenoxy)piperidin-1-yl]-2-(2H-tetrazol-5-yl)[1,3]thiazolo[5,4-d]pyrimidine

To a solution of 5-[4-(2-bromo-5-fluorophenoxy)piperidin-1-yl][1,3]thiazolo[5,4-d]pyrimidine-2-carbonitrile (100 mg, 0.230 mmol) and ammonium chloride (24.63 mg, 0.461 mmol) in DMF (2.3 mL) was added sodium azide (22.45 mg, 0.345 mmol). The reaction mixture was heated at 100° C. for 0.5 h. The mixture was cooled to RT, acidified to pH about 1 using 1N HCl and extracted with EtOAc (3×15 mL). The combined organic fractions were subsequently washed with HCl (15 mL), water (15 mL), brine (15 mL), dried over MgSO₄ and evaporated under reduced pressure. The title compound was used without further purification.

¹H NMR (500 MHz, acetone-d₆): δ 8.86 (s, 1H), 7.59 (dd, 1H), 7.11 (dd, 1H), 6.73 (td, 1H), 4.93-4.90 (m, 1H), 4.20-4.14 (m, 2H), 4.04-4.00 (m, 2H), 2.12-2.06 (m, 2H), 1.92-1.86 (m, 2H). MS (+ESI) m/z 477, 479 (MH⁺).

### Step 6: Ethyl(5-{5-[4-(2-bromo-5-fluorophenoxy)piperidin-1-yl][1,3]thiazolo[5,4-d]pyrimidin-2-yl}-1H-tetrazol-1-yl)acetate

To a solution of 5-[4-(2-bromo-5-fluorophenoxy)piperidin-1-yl]-2-(2H-tetrazol-5-yl)[1,3]thiazolo[5,4-d]pyrimidine (99 mg, 0.21 mmol) and ethyl bromoacetate (34.6 μL, 0.31 mmol) in THF (1.73 ml) was added triethylamine (57.8 μL, 0.415 mmol). The reaction mixture was refluxed for 2 h. The solvent was evaporated under reduced pressure. The residue was diluted with water (15 mL) and extracted with EtOAc (3×10 mL). The combined organic layers were washed with water (15 mL), dried over MgSO₄ and evaporated under reduced pressure. Purification by Combiflash chromatography, (SiO₂-40 g, elution with 20-50% EtOAc/Hexanes over 25 min) afforded the title compound as the less polar regioisomer.

¹H NMR (500 MHz, acetone-d₆): δ 9.06 (s, 1H), 7.60 (dd, 1H), 7.11 (dd, 1H), 6.76-6.71 (dt, 1H), 5.85 (s, 2H), 4.97-4.94 (m, 1H), 4.26 (q, 2H), 4.18-4.15 (m, 2H), 4.13-4.08 (m, 2H), 2.13-2.06 (m, 2H), 1.95-1.88 (m, 2H), 1.26 (t, 3H). MS (+ESI) m/z 563, 565 (MH⁺).

### Step 7: (5-{5-[4-(2-Bromo-5-fluorophenoxy)piperidin-1-yl][1,3]thiazolo[5,4-d]pyrimidin-2-yl}-1H-tetrazol-1-yl)acetic acid

The title compound was prepared in the same manner as described in Step 5 of Example 4 from ethyl (5-{5-[4-(2-bromo-5-fluorophenoxy)piperidin-1-yl][1,3]thiazolo[5,4-d]pyrimidin-2-yl}-1H-tetrazol-1-yl)acetate and aqueous NaOH.

¹H NMR (500 MHz, acetone-d₆): δ 8.99 (s, 1H), 7.60 (dd, 1H), 7.11 (dd, 1H), 6.74 (dt, 1H), 5.48 (s, 2H), 4.97-4.93 (m, 1H), 4.18-4.13 (m, 2H), 4.10-4.06 (m, 2H), 2.07-2.02 (m, 2H), 1.93-1.89 (m, 2H). MS (+ESI) m/z 535, 537 (MH⁺).

### Example 13

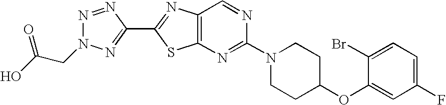

### (5-{5-[4-(2-Bromo-5-fluorophenoxy)piperidin-1-yl][1,3]thiazolo[5,4-d]pyrimidin-2-yl}-2H-tetrazol-2-yl)acetic acid

### Step 1: Ethyl(5-{5-[4-(2-bromo-5-fluorophenoxy)piperidin-1-yl][1,3]thiazolo[5,4-d]pyrimidin-2-yl}-2H-tetrazol-2-yl)acetate

The title compound was prepared in the same manner as described in Step 6 of Example 12 from 5-[4-(2-bromo-5-fluorophenoxy)piperidin-1-yl]-2-(2H-tetrazol-5-yl)[1,3]thiazolo[5,4-d]pyrimidine and ethy bromoacetate. Purification by Combiflash chromatography, (SiO₂-40 g, elution with 20-50% EtOAc/Hexanes over 25 min) afforded the title compound as the more polar regioisomer.

¹H NMR (500 MHz, acetone-d₆): δ 9.06 (s, 1H), 7.60 (dd, 1H), 7.12 (dd, 1H), 6.76-6.71 (m, 1H), 5.85 (s, 2H), 4.97-4.93 (m, 1H), 4.29 (q, 2H), 4.22-4.14 (m, 2H), 4.12-4.07 (m, 2H), 2.13-2.06 (m, 2H), 1.94-1.89 (m, 2H), 1.29 (t, 3H). MS (+ESI) m/z 563, 565 (MH⁺).

### Step 2: (5-{5-[4-(2-Bromo-5-fluorophenoxy)piperidin-1-yl][1,3]thiazolo[5,4-d]pyrimidin-2-yl}-2H-tetrazol-2-yl)acetic acid

The title compound was prepared in the same manner as described in Step 5 of Example 4 from ethyl(5-{5-[4-(2-bromo-5-fluorophenoxy)piperidin-1-yl][1,3]thiazolo[5,4-d]pyrimidin-2-yl}-2H-tetrazol-2-yl)acetate and aqueous NaOH.

¹H NMR (500 MHz, acetone-d₆): δ 9.03 (s, 1H), 7.60 (dd, 1H), 7.11 (dd, 1H), 6.73 (dt, 1H), 5.42 (s, 2H), 4.98-4.94 (m, 1H), 4.21-4.15 (m, 2H), 4.11-4.05 (m, 2H), 2.08-2.04 (m, 2H), 1.93-1.89 (m, 2H). MS (+ESI) m/z 535, 537 (MH⁺).

### Example 14

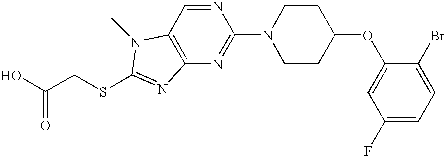

### ({2-[4-(2-Bromo-5-fluorophenoxy)piperidin-1-yl]-7-methyl-7H-purin-8-yl}thio)acetic acid

### Step 1: Ethyl({2-[4-(2-bromo-5-fluorophenoxy)piperidin-1-yl]-7-methyl-7H-purin-8-yl}thio)acetate

To a mixture of ethyl ({2-[4-(2-bromo-5-fluorophenoxy)piperidin-1-yl]-9H-purin-8-yl}thio)acetate (200 mg, 0.39 mmol) (Example 8, step 2) and iodomethane (36.8 μL, 0.59 mmol) in DMF (1.30 mL) was added K₂CO₃ (108 mg, 0.78 mmol). The mixture was stirred at RT for 4 h. The reaction mixture was diluted with EtOAc (5 ml)/NH₄Cl (10 mL) and extracted with EtOAc (2×5 mL). The organic layers were subsequently washed with aqueous saturated NH₄Cl (10 mL), water (10 mL), dried over MgSO₄, filtered and evaporated under reduced pressure. Purification by Combiflash chromatography, (SiO₂-40 g, elution with 40-60% EtOAc/Hexanes over 30 min) afforded the title compound as the more polar regioisomer.

¹H NMR (500 MHz, acetone-d₆): δ 8.57 (s, 1H), 7.61 (dd, 1H), 7.11 (dd, 1H), 6.75 (dt, 1H), 4.91-4.87 (m, 1H), 4.32 (s, 2H), 4.21 (q, 2H), 4.22-4.17 (m, 2H), 3.86-3.81 (m, 2H), 3.79 (s, 3H), 2.10-2.07 (m, 2H) 1.84-1.79 (m, 3H), 1.27 (t, 3H). MS (+ESI) m/z 524, 526 (MH⁺H).

### Step 2: ({2-[4-(2-Bromo-5-fluorophenoxy)piperidin-1-yl]-7-methyl-7H-purin-8-yl}thio)acetic acid

The title compound was prepared in the same manner as described in Step 5 of Example 4 from ethyl({2-[4-(2-bromo-5-fluorophenoxy)piperidin-1-yl]-7-methyl-7H-purin-8-yl}thio)acetate and aqueous NaOH.

¹H NMR (500 MHz, acetone-d₆): δ 8.58 (s, 1H), 7.61 (dd, 1H), 7.11 (dd, 1H), 6.73 (dt, 1H), 4.91-4.87 (m, 1H), 4.21 (s, 2H), 4.21-4.16 (m, 2H) 3.87-3.82 (m, 2H), 3.80 (s, 3H), 2.06-1.99 (m, 2H), 1.86-1.79 (m, 2H). MS (+ESI) m/z 496, 498 (MH⁺).

### Example 15

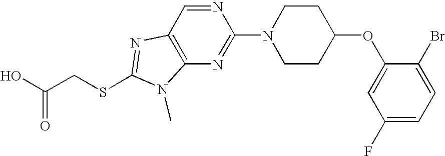

### ({2-[4-(2-Bromo-5-fluorophenoxy)piperidin-1-yl]-9-methyl-9H-purin-8-yl}thio)acetic acid

### Step 1: Ethyl({2-[4-(2-bromo-5-fluorophenoxy)piperidin-1-yl]-9-methyl-9H-purin-8-yl}thio)acetate

The title compound was prepared in the same manner as described in Step 1 of Example 14 from ethyl ({2-[4-(2-bromo-5-fluorophenoxy)piperidin-1-yl]-9H-purin-8-yl}thio)acetate (Example 8, step 2) and iodomethane. The product was obtained as the less polar regioisomer.

¹H NMR (500 MHz, acetone-d₆): δ 8.47 (s, 1H), 7.62 (dd, 1H), 7.12 (dd, 1H), 6.75 (td, 1H), 4.92-4.87 (m, 1H), 4.24 (s, 2H), 4.20 (q, 2H), 4.22-4.17 (m, 2H) 3.95-3.89 (m, 2H), 3.65 (s, 3H), 2.12-2.05 (m, 2H), 1.87-1.81 (m, 2H), 1.26 (t, 3H). MS (+ESI) m/z 524, 526 (MH⁺).

### Step 2: ({2-[4-(2-Bromo-5-fluorophenoxy)piperidin-1-yl]-9-methyl-9H-purin-8-yl}thio)acetic acid

The title compound was prepared in the same manner as described in Step 5 of Example 4 from ethyl({2-[4-(2-bromo-5-fluorophenoxy)piperidin-1-yl]-9-methyl-9H-purin-8-yl}thio)acetate and aqueous NaOH.

¹H NMR (500 MHz, acetone-d₆): δ 8.49 (s, 1H), 7.62 (dd, 1H), 7.12 (dd, 1H), 6.77-6.72 (m, 1H), 4.92-4.89 (m, 1H), 4.18 (s, 2H), 4.22-34.17 (m, 2H), 3.94-3.89 (m, 2H), 3.65 (s, 3H), 2.12-2.05 (m, 2H), 1.89-1.81 (m, 2H). MS (+ESI) m/z 496, 498 (MH⁺).

### Example 16

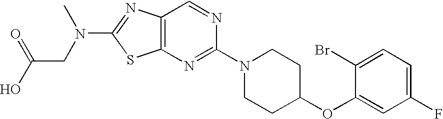

### [{5-[4-(2-Bromo-5-fluorophenoxy)piperidin-1-yl][1,3]thiazolo[5,4-d]pyrimidin-2-yl}(methyl)amino]acetic acid

### Step 1: 2-Methoxyethyl[{5-[4-(2-bromo-5-fluorophenoxy)piperidin-1-yl][1,3]thiazolo[5,4-d]pyrimidin-2-yl}(methyl)amino]acetate

The title compound was prepared in the same manner as described in Step 1 of Example 14 from 2-methoxyethyl({5-[4-(2-bromo-5-fluorophenoxy)piperidin-1-yl][1,3]thiazolo[5,4-d]pyrimidin-2-yl}amino)acetate (Example 7, step 2) and iodomethane.

¹H NMR (500 MHz, acetone-d₆): δ 8.40 (s, 1H), 7.61 (dd, 1H), 7.11 (dd, 1H), 6.75 (td, 1H), 4.92-4.87 (m, 1H), 4.45 (s, 2H), 4.28 (t, 2H), 4.14-4.07 (m, 2H), 3.90-3.84 (m, 2H), 3.61 (t, 2H), 3.32 (s, 3H), 3.27 (s, 3H), 2.10-2.04 (m, 2H) 1.89-1.81 (m, 2H). MS (+ESI): m/z 554, 556 (MH⁺).

### Step 2: [{5-[4-(2-Bromo-5-fluorophenoxy)piperidin-1-yl][1,3]thiazolo[5,4-d]pyrimidin-2-yl}(methyl)amino]acetic acid

The title compound was prepared in the same manner as described in Step 5 of Example 4 from 2-methoxyethyl[{5-[4-(2-bromo-5-fluorophenoxy)piperidin-1-yl][1,3]thiazolo[5,4-d]pyrimidin-2-yl}(methyl)amino]acetate and aqueous NaOH.

¹H NMR (400 MHz, acetone-d₆): δ 8.39 (s, 1H), 7.62 (dd, 1H), 7.12 (dd, 1H), 6.75 (td, 1H), 4.92-4.88 (m, 1H), 4.36 (s, 2H), 4.15-4.09 (m, 2H), 3.90-3.83 (m, 2H), 3.28 (s, 3H), 2.09-2.06 (m, 2H), 1.89-1.81 (m, 2H). MS (+ESI) m/z 496, 498 (MH⁺).

### Example of a Pharmaceutical Formulation

As a specific embodiment of an oral composition of a compound of the present invention, 50 mg of the compound of any of the Examples is formulated with sufficient finely divided lactose to provide a total amount of 580 to 590 mg to fill a size O hard gelatin capsule.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of structural formula I: 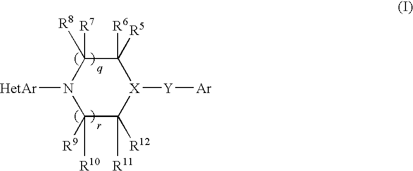
or a pharmaceutically acceptable salt thereof; wherein
HetAr is a fused heteroaromatic ring selected from the group consisting of: 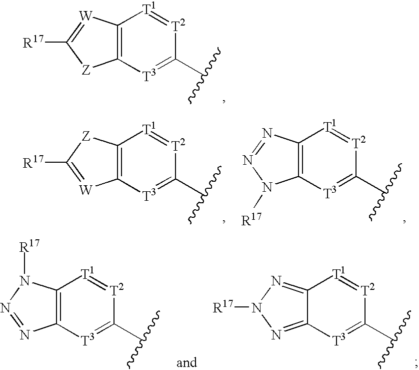
wherein W is N or CR¹⁶;
Z is O, S, or NR¹⁵;
T¹, T², and T³ are each independently N or CR¹⁶, with the proviso that at least one of T¹, T², and T³ is N;
q is 0 or 1;
r is 0 or 1;
X—Y is N—C(O), CR¹⁴—O, CR¹⁴—S(O)₀₋₂, or CR¹³—CR¹R²;
Ar is phenyl, naphthyl, or heteroaryl optionally substituted with one to five R³ substituents;
R¹ and R² are each independently hydrogen or C₁₋₃ alkyl, wherein alkyl is optionally substituted with one to three substituents independently selected from fluorine and hydroxy;
each R³ is independently selected from the group consisting of:
C₁₋₆ alkyl,
C₂₋₆ alkenyl,
(CH₂)ₙ-phenyl,
(CH₂)ₙ-naphthyl,
(CH₂)ₙ-heteroaryl,
(CH₂)ₙ-heterocyclyl,
(CH₂)ₙC₃-₇ cycloalkyl,
halogen,
nitro,
(CH₂)ₙOR⁴,
(CH₂)ₙN(R⁴)₂,
(CH₂)ₙC≡N,
(CH₂)ₙCO₂R⁴,
(CH₂)ₙNR⁴SO₂R⁴
(CH₂)ₙSO₂N(R⁴)₂,
(CH₂)ₙS(O)₀₋₂R⁴,
(CH₂)ₙNR⁴C(O)N(R⁴)₂,
(CH₂)ₙC(O)N(R⁴)₂,
(CH₂)ₙNR⁴C(O)R⁴,
(CH₂)ₙNR⁴CO₂R⁴,
(CH₂)ₙC(O)R⁴,
O(CH₂)ₙC(O)N(R⁴)₂,
(CH₂)ₛ—Z—(CH₂)ₜ-phenyl,
(CH₂)ₛ—Z—(CH₂)ₜ-naphthyl,
(CH₂)ₛ—Z—(CH₂)ₜ-heteroaryl,
(CH₂)ₛ—Z—(CH₂)ₜ-heterocyclyl,
(CH₂)ₛ—Z—(CH₂)ₜ—C₃₋₇ cycloalkyl,
(CH₂)ₛ—Z—(CH₂)ₜ—OR⁴,
(CH₂)ₛ—Z—(CH₂)ₜ—N(R⁴)₂,
(CH₂)ₛ—Z—(CH₂)ₜ—NR⁴SO₂R⁴,
(CH₂)ₛ—Z—(CH₂)ₜ—C≡N,
(CH₂)ₛ—Z—(CH₂)ₜ—CO₂R⁴,
(CH₂)ₛ—Z—(CH₂)ₜ—SO₂N(R⁴)₂,
(CH₂)ₛ—Z—(CH₂)ₜ—S(O)₀₋₂R⁴,
(CH₂)ₛ—Z—(CH₂)ₜ—NR⁴C(O)N(R⁴)₂,
(CH₂)ₛ—Z—(CH₂)ₜ—C(O)N(R⁴)₂,
(CH₂)ₛ—Z—(CH₂)ₜ—NR⁴C(O)R⁴,
(CH₂)ₛ—Z—(CH₂)ₜ—NR⁴CO₂R⁴,
(CH₂)ₛ—Z—(CH₂)ₜ—C(O)R⁴,
CF₃,
CH₂CF₃,
OCF₃, and
OCH₂CF₃;
in which phenyl, naphthyl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy; and wherein any methylene (CH₂) carbon atom in R³ is optionally substituted with one to two groups independently selected from fluorine, hydroxy, and C₁₋₄ alkyl; or two substituents when on the same methylene (CH₂) group are taken together with the carbon atom to which they are attached to form a cyclopropyl group;
Z is O, S, or NR⁴;
each R⁴ is independently selected from the group consisting of
hydrogen,
C₁₋₆ alkyl,
(CH₂)ₘ-phenyl,
(CH₂)ₘ-heteroaryl,
(CH₂)ₘ-naphthyl, and
(CH₂)ₘC₃-₇ cycloalkyl;
wherein alkyl, phenyl, heteroaryl, and cycloalkyl are optionally substituted with one to three groups independently selected from halogen, C₁₋₄ alkyl, and C₁₋₄ alkoxy; or two R⁴ groups together with the atom to which they are attached form a 4- to 8-membered mono- or bicyclic ring system optionally containing an additional heteroatom selected from O, S, NH, and NC₁₋₄ alkyl;
R⁵, R⁶, R⁷, R⁸, R⁹, R¹⁰, R¹¹, and R¹² are each independently hydrogen, fluorine, or C₁₋₃ alkyl, wherein alkyl is optionally substituted with one to three substituents independently selected from fluorine and hydroxy;
R¹³ is hydrogen, C₁₋₃ alkyl, fluorine, or hydroxy;
each R¹⁴ is hydrogen or C₁₋₃ alkyl;
R¹⁵ is selected from the group consisting of hydrogen, C₁₋₄ alkyl, C₁₋₄ alkylcarbonyl, aryl-C₁₋₂ alkylcarbonyl, arylcarbonyl, C₁₋₄ alkylaminocarbonyl, C₁₋₄ alkylsulfonyl, arylsulfonyl, aryl-C₁₋₂ alkylsulfonyl, C₁₋₄ alkyloxycarbonyl, aryloxycarbonyl, and aryl-C₁₋₂ alkyloxycarbonyl;
R¹⁶ is hydrogen, amino, halogen, or C₁₋₃ alkyl optionally substituted with one to five fluorines;
R¹⁷ is selected from the group consisting of:
—(CH₂)ᵥC(O)R^{a};
—O(CH₂)_{w}C(O)R^{a},
—S(CH₂)_{w}C(O)R^{a},
—NH(CH₂)_{w}C(O)R^{a},
—NCH₃(CH₂)_{w}C(O)R^{a}, 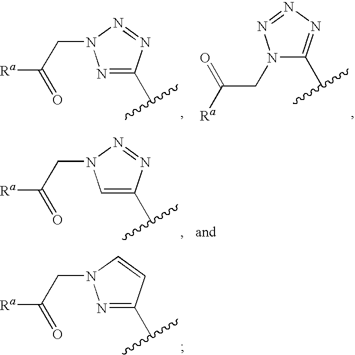
R^{a} is —OH, —OC₁₋₄ alkyl, —NH₂, —NHSO₂C₁₋₄ alkyl, —NHSO₂C₃₋₆ cycloalkyl, or —NHSO₂CH₂C₃₋₆ cycloalkyl;
each m is independently an integer from 0 to 2;
each n is independently an integer from 0 to 2;
each s is independently an integer from 1 to 3;
each t is independently an integer from 1 to 3;
v is an integer from 1 to 3; and
each w is an integer from 1 to 2.

2. The compound of claim 1 wherein q and r are both 1.

3. The compound of claim 1 wherein X—Y is CR¹⁴—O.

4. The compound of claim 3 wherein R¹⁴ is hydrogen and Ar is phenyl substituted with one to three R³ substituents.

5. The compound of claim 1 wherein R⁵, R⁶, R⁷, R⁸, R⁹, R¹⁰, R¹¹, and R¹² are each hydrogen.

6. The compound of claim 1 wherein T¹ is CR¹⁶, and T² and T³ are each N; or T² is CR¹⁶, and T¹ and T³ are each N.

7. The compound of claim 6 wherein R¹⁶ is hydrogen.

8. The compound of claim 1 wherein HetAr is

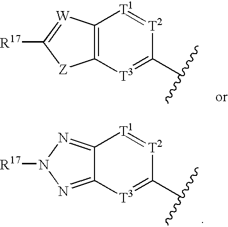

9. The compound of claim 8 wherein T¹ is CH, and T² and T³ are each N; or T² is CH, and T¹ and T³ are each N.

10. The compound of claim 9 wherein Z is S, and W is NH.

11. The compound of claim 1 wherein R^{a} is OH or —OC₁₋₄ alkyl.

12. The compound of claim 11 wherein v is 2 and each w is 1.

13. The compound of claim 1 wherein Ar is phenyl substituted with one to two substituents independently selected from the group consisting from C₁₋₄ alkyl, halogen, and CF₃.

14. The compound of claim 1 wherein
X—Y is CH—O;
q and r are each 1;
R⁵, R⁶, R⁷, R⁸, R⁹, R¹⁰, R¹¹, and R¹² are each hydrogen;
Ar is phenyl substituted with one to three R³ substituents;
HetAr is 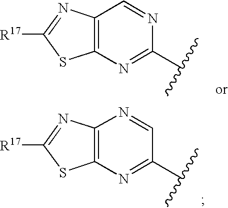
R¹⁷ is selected from the group consisting of:
—(CH₂)₂C(O)R^{a};
—OCH₂C(O)R^{a},
—SCH₂C(O)R^{a},
—NHCH₂C(O)R^{a}, and 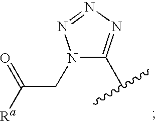
and
R^{a} is —OH or —OC₁₋₄ alkyl.

15. The compound of claim 14 wherein Ar is phenyl substituted with one to two substituents independently selected from the group consisting from C₁₋₄ alkyl, halogen, and CF₃.

16. The compound of claim 15 which is selected from the group consisting of: 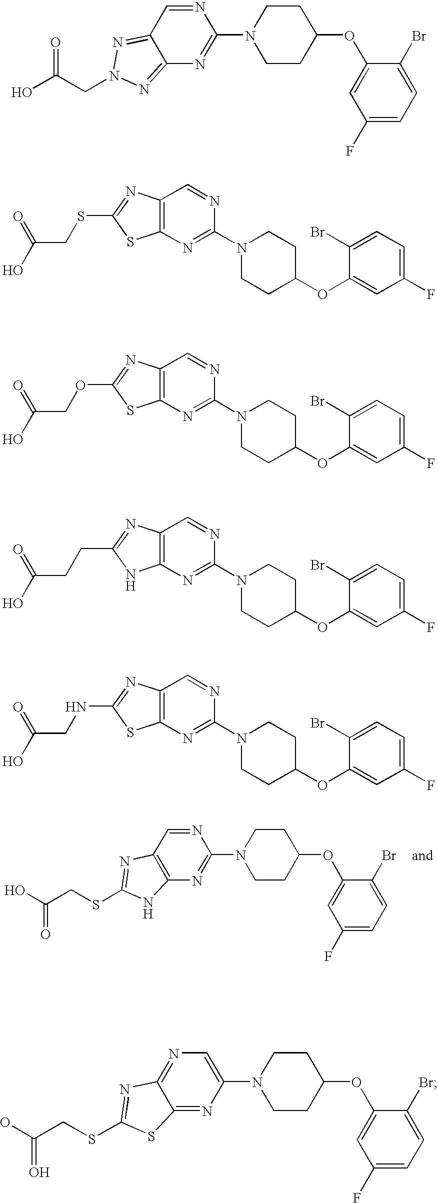
or a pharmaceutically acceptable salt thereof.

17. A pharmaceutical composition comprising a compound in accordance with claim 1 in combination with a pharmaceutically acceptable carrier.

18-21. (canceled)

22. A method for treating non-insulin dependent (Type 2) diabetes, insulin resistance, hyperglycemia, a lipid disorder, obesity, and fatty liver disease in a mammal in need thereof which comprises the administration to the mammal of a therapeutically effective amount of a compound of claim 1.

23. The method of claim 22 wherein said lipid disorder is selected from the group consisting of dyslipidemia, hyperlipidemia, hypertriglyceridemia, atherosclerosis, hypercholesterolemia, low HDL, and high LDL.
